SEQUENCE LISTING (1 sequence)

SEQ ID NO: 1 - DNA, 15954 bp, Murine
caagcttcagaatgtgcttcaggatgggactgaccttggtcacacattgcctattaggtagagtctagtctttattatatgaatgaagattatacaaagggttcgaagtcttacacgaagtcctaccctgactggaaccagtgtgtaacggataatccatctcagatcagaaataatatacttacttctaatatgtttcctacggttgtgtgtaattgtgccttaaaatgtatcagggtcggatgtgaaatcatcctactgaaacccaaaatacataaaagaaaaaaaaatgagtttgttatgccaacacacattaacacggaattttacatagtcccagcctacactttagtaggatgactttgggttttatgtattttctttttttttactcaaacaacttattttaggtatcagcctagcagtaaaaccgaccagaagccatgcacaaaactacatccccagagaaagacttgtcccttcccctccctgtcatctcagaataaaatccatagtcggatcgtcattttggctggtcttcggtacgtgttttgatgtaggggtctctttctgaacagggaaggggagggacagtagagtccatgaacatggttcaagacagcgcctttctagccaagctgatgaagagtgctgacacctttgagttgaagtatgacttttcctgtgagctgtaccgattggtacttgtaccaagttctgtcgcggaaagatcggttcgactacttctcacgactgtggaaactcaacttcatactgaaaaggacactcgacatggctaagtccacgtattcagcttttcccaggggagttcctgtgtcagaaaggagtctggctcgtgctggcttttactacactggtgccaatgacaaggtcaagtgccaggtgcataagtcgaaaagggtcccctcaaggacacagtctttcctcagaccgagcacgaccgaaaatgatgtgaccacggttactgttccagttcacgttctgctgtggcctgatgctagacaactggaaacaaggggacagtcccatggagaagcacagaaagttgtaccccagctgcaactttgtacagactttgaaagacgacaccggactacgatctgttgacctttgttcccctgtcagggtacctcttcgtgtctttcaacatggggtcgacgttgaaacatgtctgaaactatccagccaacagtctggaagctagtcctcggccttctcttccttccacggcgatgagcaccatgcctttgagctttgcaagttctgagaatactggctataggtcggttgtcagaccttcgatcaggagccggaagagaaggaaggtgccgctactcgtggtacggaaactcgaaacgttcaagactcttatgaccgattttcagtggctcttactcgagctttccctcagaccctgtgaacttccgagcaaatcaagattgtcctgctttgagcacaagtccctaccactttgcaatgaaagtcaccgagaatgagctcgaaagggagtctgggacacttgaaggctcgtttagttctaacaggacgaaactcgtgttcagggatggtgaaacgttacaacacagagaaggccagattactcacctatgaaacatggccattgtcttttctgtcaccagcaaagctggccaaagcaggcttctactacataggacctgttgtgtctcttccggtctaatgagtggatactttgtaccggtaacagaaaagacagtggtcgtttcgaccggtttcgtccgaagatgatgtatcctggacgagatagagtggcctgctttgcgtgcgatgggaaactgagcaactgggaacgtaaggatgatgctatgtcagagcaccagaggcatttccccagctgtccctctatctcaccggacgaaacgcacgctaccctttgactcgttgacccttgcattcctactacgatacagtctcgtggtctccgtaaaggggtcgacagggttcttaaaagacttgggtcagtctgcttcgagatacactgtctctaacctgagcatgcagacacacgcagcccgtattagaacattctctaactggcctcaagaattttctgaacccagtcagacgaagctctatgtgacagagattggactcgtacgtctgtgtgcgtcgggcataatcttgtaagagattgaccggatctagtgcactagttcattcccaggaacttgcaagtgcgggcttttattatacaggtaagaaacattctgagaattataaagatctgtcatgtgttatacagatcacgtgatcaagtaagggtccttgaacgttcacgcccgaaaataatatgtccattctttgtaagactcttaatatttctagacagtacacaatatgacccaaaagggcaaattttatatgtttataagttttcgttcaaaatgaattttaggacacagtgatgatgtcaagtgtttttgctgtgatggtgggctgatgggttttcccgtttaaaatatacaaatattcaaaagcaagttttacttaaaatcctgtgtcactactacagttcacaaaaacgacactaccacccgactggtgctgggaatctggagatgacccctgggtggaacatgccaagtggtttccaaggtaacggctttgaaattattgcatcttttatgttttcatgagataccacgacccttagacctctactggggacccaccttgtacggttcaccaaaggttccattgccgaaactttaataacgtagaaaatacaaaagtactctatcattatcttcttttatagcaagtgtgcctcttaagtcctacagattttattttattcttatgtatatgagtgttttgcatatatgtctgtatactgtttggtaatagaagaaaatatcgttcacacggagaattcaggatgtctaaaataaaataagaatacatatactcacaaaacgtatatacagacatatgacaaactgtgcagtgactgcagaggccaggagaggacactggagcctctgtgactagacttctagattgttgtgggtcaacatgtgggagctgggaattaaacctgacacgtcactgacgtctccggtcctctcctgtgacctcggagacactgatctgaagatctaacaacacccagttgtacaccctcgacccttaatttggacggttctctgaaagaacagccagtgctctgtcttggtgagccttctcttaagctctattacttcagactttaaacattataaagaatataaagaacattcaccaagagactttcttgtcggtcacgagacagaaccactcggaagagaattcgagataatgaagtctgaaatttgtaatatttcttatatttcttgtaagtcattgatttttttaagtgctttgcattgtgatagttaaaaacttttttcttttccatttatttgttttatgtgtatgtggtgtgaaggtcaggggataacgtaactaaaaaaattcacgaaacgtaacactatcaatttttgaaaaaagaaaaggtaaataaacaaaatacacatacaccacacttccagtcccctattgttttgagggccagttctcacctgttacccttgcgattggactcaggttactaggcctggtggaaagaacttttgctagctgagccatctcaccagcccctaaaactcccggtcaagagtggacaatgggaacgctaacctgagtccaatgatccggaccacctttcttgaaaacgatcgactcggtagagtggtcggggaccccactgtgaatacacaagcactgtgagcctgctttgatcctgccatgagtcactttatagttaaatttgataatttacctgtattacctagggattgaggggtgacacttatgtgttcgtgacactcggacgaaactaggacggtactcagtgaaatatcaatttaaactattaaatggacataatggatccctaactcaacctccttagtagaggagtcataacgaatgtcacattctttccttagagtgcatctatacatgtttttagcccataaaattgtcatggagggctggtggttggaggaatcatctcctcagtattgcttacagtgtaagaaaggaatctcacgtagatatgtacaaaaatcgggtattttaacagtacctcccgaccacagatggctcagtgggtaggagcacccgactgctcttccgaaggtccggagttcaaatcccagcaaccacatggtggctcacaaccatccgtaatgagatctctaccgagtcacccatcctcgtgggctgacgagaaggcttccaggcctcaagtttagggtcgttggtgtaccaccgagtgttggtaggcattactctagtgactccctcttctggagtgtctgaggacagctacagtgtacttacatttaataaataaataaatctttaaaaatataaaaaaaaaaaaaaattgtcatgactgagggagaagacctcacagactcctgtcgatgtcacatgaatgtaaattatttatttatttagaaatttttatatttttttttttttttaacagtacgagatgaccagatggcttatttaggagactccatatttgcttaagggtatttttaggaacctttgaagagtagagagcagtgaggagctctgaggagccactctactggtctaccgaataaatcctctgaggtataaacgaattcccataaaaatccttggaaacttctcatctctcgtcactcctcgagactcctcggtgctggagggtggtggctgaggggctcctcatggccactgcttggtggctcagctgcttactcagtccccagttcctgtagctttggttactgaaggaccacgacctcccaccaccgactccccgaggagtaccggtgacgaaccaccgagtcgacgaatgagtcaggggtcaaggacatcgaaaccaatgacttcctggtgatgcttgctgcttgtttccaggagttccactgcatttacacacacaaaccacacacagacagagaaatgtacacaagtaaaaaatcttcatgcatagagctacgaacgacgaacaaaggtcctcaaggtgacgtaaatgtgtgtgtttggtgtgtgtctgtctctttacatgtgttcattttttagaagtacgtatctctcagtgtgcgctgagctggggcttatttctcttccaacattaagaatctgacctccagtagctcctccccagggcgcttagccagcttccacctgagatgagtcacacgcgactcgaccccgaataaagagaaggttgtaattcttagactggaggtcatcgaggaggggtcccgcgaatcggtcgaaggtggactctacgacatccagacatgggtttgttattaagtttcatttcactttctaaaattaagattaatttttgatcgtaaaacatgttatgaagctgatgatgggcaatctgtaggtctgtacccaaacaataattcaaagtaaagtgaaagattttaattctaattaaaaactagcattttgtacaatacttcgactactacccgttagctgtcttactaagtaattcctgatgttgatgctttaggtgtgagtacttgctcagaatcaaaggccaagaatttgtcagccaagttcaagctggctatccgacagaatgattcattaaggactacaactacgaaatccacactcatgaacgagtcttagtttccggttcttaaacagtcggttcaagttcgaccgatagctcatctacttgagcaggtaggaggcctccctgttttatttttaattattatttaaaaaaaattattggctgggcatggtggcgcacgcctttaatcccagagtagatgaactcgtccatcctccggagggacaaaataaaaattaataataaattttttttaataaccgacccgtaccaccgcgtgcggaaattagggtgcactcaggaggcagaggcaggtggatttctgagtttgaggccagcctggtctacaaagtgagttccaggacagccagagagagaaaccctgtctcgaaacgtgagtcctccgtctccgtccacctaaagactcaaactccggtcggaccagatgtttcactcaaggtcctgtcggtctctctctttgggacagagctttaaaaaattatttgtttgcaaaaattatttttaaaaatttatttgtaaaaataaatcagaataatatttcagtgaaaagtgaaagttgttttaaaacacgtttttttaataaacaaacgtttttaataaaaatttttaaataaacatttttatttagtcttattataaagtcacttttcactttcaacaaaattttgtgcacctcctttcttctcacaaatgtcaacctaagtgtgcaaggcacttctcttttgagaaaaacagatctgcacctattagccacatggaagatagtatttgtggaggaaagaagagtgtttacagttggattcacacgttccgtgaagagaaaactctttttgtctagacgtggataatcggtgtaccttctatcataaacatctatttctctttagctgtcatctaatcagctaataataaattaaactctgaaattaataaagtaaataatgcatttgaggaaaacaactttactgtgcaagataaagagaaatcgacagtagattagtcgattattatttaatttgagactttaattatttcatttattacgtaaactccttttgttgaaatgacacgtaccttttcttaagtgacttgagaaagcatttctcctcagaaagctaatgagatcttatttaattgtctgaggttcaaaaacgcatttgccatggccagcatggaaaagaattcactgaactctttcgtaaagaggagtctttcgattactctagaataaattaacagactccaagtttttgcgtaaacggtaccggtcgtgacctgactgtggactgtgtcccagcagatgccatggccagcagacctgactgtggactgtgtcccagcagatgccatggccagcagacctgactgtggactggactgacacctgacacagggtcgtctacggtaccggtcgtctggactgacacctgacacagggtcgtctacggtaccggtcgtctggactgacacctctgtgtcccagcagatgccatggccagcagacctgactgtggactgtgtcccagcagatgccatggccagcagacctgactgtggactgtgtcccagcaggacacagggtcgtctacggtaccggtcgtctggactgacacctgacacagggtcgtctacggtaccggtcgtctggactgacacctgacacagggtcgtcatgccatggccagcagacctgactgtggactgtgtcccagcagatgccatggccagcagacctgactgtggactgtgtcccagcagatgccatggccagctacggtaccggtcgtctggactgacacctgacacagggtcgtctacggtaccggtcgtctggactgacacctgacacagggtcgtctacggtaccggtcgagacctgactgtggactgtgtcccagcagatgccatggccagcagacctgactgtggactgtgtcccagcagatgactagggaacacgagatttgcagattctggactgacacctgacacagggtcgtctacggtaccggtcgtctggactgacacctgacacagggtcgtctactgatcccttgtgctctaaacgtctactaggatcaacctgcagcctcccaggaaacccagcctgtcctgcccaatgaggacagacttagctcactggagcacagtctccataagaagctgttagttgatcctagttggacgtcggagggtcctttgggtcggacaggacgggttactcctgtctgaatcgagtgacctcgtgtcagaggtattcttcgacaatcaatagagcctcattattcaaatccctggtgaatgtaaatgaaacattgcttctgactggggaaatgccctgatgatgacaaagtcggttggtggtaggttaaatctcggagtaataagtttagggaccacttacatttactttgtaacgaagactgacccctttacgggactactactgtttcagccaaccaccatccaattatggagcaggtctgttggtcattttcagtgcagtgctaggatgcagcttacattttaacatgtgtattctttcctgtagctattatctacgtcagactcctacctcgtccagacaaccagtaaaagtcacgtcacgatcctacgtcgaatgtaaaattgtacacataagaaaggacatcgataatagatgcagtctgaggccagaagatgagaatgcagacgcagcaagtatgtataataatcataattcctgcattacactgctctgttttgcataaggttgaagggaagctgagtccaggtcttctactcttacgtctgcgtcgttcatacatattattagtattaaggacgtaatgtgacgagacaaaacgtattccaacttcccttcgactcaggtgctgtaatccagcatttggaaagcagaagtggaaggctgatgtgctcaaggccctattgaattatgtgagaccttgtcctagaaatacacatgattggaacgacattaggtcgtaaacctttcgtcttcaccttccgactacacgagttccgggataacttaatacactctggaacaggatctttatgtgtactaaccttggttatcataggaaagaaatatgcatgttgatttctaatgtatataagcaacaagagttgcttggagaaatttatttatttatttaaaagatttatttatccaatagtatcctttctttatacgtacaactaaagattacatatattcgttgttctcaacgaacctctttaaataaataaataaattttctaaataaatatattatatctaagtacactgtagctgtcttcagatgcaccaaaagagggcatcagatctcattacagatggttgtgagccaccatgtggttgctgggaatataatatagattcatgtgacatcgacagaagtctacgtggttttctcccgtagtctagagtaatgtctaccaacactcggtggtacaccaacgacccttatgaactcaggacctttggaagagcagtcagtgctcttaaccactgagccatctctccagcccgcttggagaaatttaaaaaacagcataaatatctagttacttgagtcctggaaaccttctcgtcagtcacgagaattggtgactcggtagagaggtcgggcgaacctctttaaattttttgtcgtatttatagatcaatatgcttcaggcctcatagattgatcagtgtatgtaagtgtgtatcttgggtctctatgtataattgttttttatgctcctgggggttgaacctaggaccatacgaagtccggagtatctaactagtcacatacattcacacatagaacccagagatacatattaacaaaaaatacgaggacccccaacttggatcctggtcaggcatgccaatcataacctctttccctagctccaccccatagcagtgggcatctacactttaagcaaactacattcatctagccttaccctgttgttagtccgtacggttagtattggagaaagggatcgaggtggggtatcgtcacccgtagatgtgaaattcgtttgatgtaagtagatcggaatgggacaacaagggcagttgtggaaatacgtatactgtgccttctattgctggcatactcttcctgtccgtagaaagggtagaacacagaattcgaattttttcactgggccccgtcaacacctttatgcatatgacacggaagataacgaccgtatgagaaggacaggcatctttcccatcttgtgtcttaagcttaaaaaagtgacccggcagattgccaatggaacttacttgttacttagacccgagcggatggagcagtcgtttgaataggtgccttgaccttatgttttagtgctcagtgcaatacgtctaacggttaccttgaatgaacaatgaatctgggctcgcctacctcgtcagcaaacttatccacggaactggaatacaaaatcacgagtcacgttatcattttaatcatttgtttactttcatgtatgtgcattggtgttttgcctgctagtatgtttgtgtgaagtgtcaggtcccctggaactggagttaaagacgtaaaattagtaaacaaatgaaagtacatacacgtaaccacaaaacggacgatcatacaaacacacttcacagtccaggggaccttgacctcaatttctgagttgagagctgtcactgaatgctaggaattgaacctgggtcctctggaagagcagtcagtacccttaaccactgagtcatctctctagcccataagccctcaactctcgacagtgacttacgatccttaacttggacccaggagaccttctcgtcagtcatgggaattggtgactcagtagagagatcgggtattcgggatatggccctgttgatttttgttgttgatgttgttcaaaggaaagaataaaaaacaagataagcatgtgacttctcatgagctaatgccattggggacagtataccgggacaactaaaaacaacaactacaacaagtttcctttcttattttttgttctattcgtacactgaagagtactcgattacggtaacccctgtcataatgcccagctaatagaacagggtctgaaggagaacgtgaagagacaactgtctaaatctggggaacacaaaaggctttctaggaggccaaatgtggctattacgggtcgattatcttgtcccagacttcctcttgcacttctctgttgacagatttagaccccttgtgttttccgaaagatcctccggtttacaccgagcatctgtctggggcagggccagcaatctaagtacttggagggttaaggcacagggtcatacattcctagccagccggggttatacagtgagtggccagtcgtagacagaccccgtcccggtcgttagattcatgaacctcccaattccgtgtcccagtatgtaaggatcggtcggccccaatatgtcactcaccggtccctggataacctactgaaaccttgtcttaaaagaacaaaccaaggttggcagatgactcagcccaggttgtagcccctggactcacatggtcgaggggaaggacctattggatgactttggaacagaattttcttgtttggttccaaccgtctactgagtcgggtccaacatcggggacctgagtgtaccagctccccttagctgactcttgaaagttgtcctctgactacccacgtgccatagtgcacacattccttctcaagaaagaaagaaagaaagaaagaaagaaagaaagaaagtcgactgagaactttcaacaggagactgatgggtgcacggtatcacgtgtgtaaggaagagttctttctttctttctttctttctttctttctttctttcaaagaaagaaagagagaaagaaagacccataaaataaagcaaactaaaaactgaaaaaaaaaaaaggcactctggaagaaatacttatcttagtagagagtttctttctttctctctttctttctgggtattttatttcgtttgatttttgacttttttttttttccgtgagaccttctttatgaatagaatcatctctccccaagaacaaatggaatcagttaactgacagggcagagcatattctgggcaagacaacatcagaggctaagcaacatggaatagggacattcaaagaacgggttcttgtttaccttagtcaattgactgtcccgtctcgtataagacccgttctgttgtagtctccgattcgttgtaccttatccctgtaagtttcttgagagcaaagattttgtctagccagaagagggagctgcagacagggaattatgaggccagcttgctgaatcaaaagctatcagttcctgagttccagtgcatctcgtttctaaaacagatcggtcttctccctcgacgtctgtcccttaatactccggtcgaacgacttagttttcgatagtcaaggactcaaggtcacgtgagtgcaaggggcaagtgcctgaggctgcttatggcctggaagtagcaagaggtgaggcagagagttagatcagaatgtaaccacagttagatttgagatctcacgttccccgttcacggactccgacgaataccggaccttcatcgttctccactccgtctctcaatctagtcttacattggtgtcaatctaaactctattttatggaaggctatggcgttcagtttggggagtatgacaccaggtttgcactttaagtggttgggttgtagtgttaagtaagctggaaagggggtgggaaaataccttccgataccgcaagtcaaacccctcatactgtggtccaaacgtgaaattcaccaacccaacatcacaattcattcgacctttcccccaccctagcactctgggacactggggtagccaggctgaggcaagatccttgtctatatgctgagtccttgaccctcacaaacaaacaaaaagaatgaaagaaaatatcgtgagaccctgtgaccccatcggtccgactccgttctaggaacagatatacgactcaggaactgggagtgtttgtttgtttttcttactttcttttatacccaaccataaattaaagcgtgaaaccatgccacttgcaagaaatggaaagaactagaaatatcaaagtatgtgctgcgtgaatatgaatatacaattatgggttggtatttaatttcgcactttggtacggtgaacgttctttacctttcttgatctttatagtttcatacacgacgcacttatacttatatgttaaaaaacattgtagacaactagtaaatacaaacaaaaggaagcaaatatgtagaagtatatatttgctatagaaagcacattagtttattgattatagagaattttgtaacatctgttgatcatttatgtttgttttccttcgtttatacatcttcatatataaacgatatctttcgtgtaatcaaataactaatatctctttgaagcccctgattctagtggaatgcctgggcattctctgctctctcctcttgagtagaactaatcacagacttgaatctaaaggcaatcgtattttgtaacttcggggactaagatcaccttacggacccgtaagagacgagagaggagaactcatcttgattagtgtctgaacttagatttccgttagcataaaacatttcttacttcaatggctgaatatatgtcattttaaagttagcaaaatgcatattgtatagtacctatttattttttatttttaatgggtttgatattgctaagaatgaagttaccgacttatatacagtaaaatttcaatcgttttacgtataacatatcatggataaataaaaaataaaaattacccaaactataacgaataactttctatttaaaaagtatatgaagccagatatgttggtttgtgcctagcactttctgaggcaggagaattggcatgaatttgagatcagcttggatattgaaagataaatttttcatatacttcggtctatacaaccaaacacggatcgtgaaagactccgtcctcttaaccgtacttaaactctagtcgaacctatatataataagcttgaggctagactggcctagaaggattcctaaaagaacaaccacaacaaaaagatgcatgggcgctgctaatatctgttaaatcccctatatattattcgaactccgatctgaccggatcttcctaaggattttcttgttggtgttgtttttctacgtacccgcgacgattatagacaatttaggggtgtccatatagtcgtgcattttggccctggagaaagttcggaagatgtcgtcatgatgagcacgcctgtggttaaagcagccttggaaatgggcttcagtacaggtatatcagcacgtaaaaccgggacctctttcaagccttctacagcagtactactcgtgcggacaccaatttcgtcggaacctttacccgaagtcaaggagcctggtgagacagacggttcagtggcagatcctggccactggtgagaactacaggaccgtcagtgacctcgttataggcttactcgatgcagaagtcctcggaccactctgtctgccaagtcaccgtctaggaccggtgaccactcttgatgtcctggcagtcactggagcaatatccgaatgagctacgtcttcacgagatgagagaggagcagatggagcaggcggccgaggaggaggagtcaggtacagagtaacaaggcctttccctggagagatgtgctgtcgcccacgctgctctactctctcctcgtctacctcgtccgccggctcctcctcctcagtccatgtctcattgttccggaaagggacctctctacacgacagcgggtgcggaattcgaaaaagatgcccatctactcagcctggcctgccttccttcctttctttctttctttctttctttctttctttctttctttctttctttctttccttaagctttttctacgggtagatgagtcggaccggacggaaggaaggaaagaaagaaagaaagaaagaaagaaagaaagaaagaaagaaagaaagaaagtttctttctttctttctttctttcttttttaaaatgtatttattattgtaaataagtacactgtcgctgtcttcagacacaccagaagagggcatctgataaagaaagaaagaaagaaagaaagaaaaaattttacataaataataacatttattcatgtgacagcgacagaagtctgtgtggtcttctcccgtagactacccattacagatggttgtgagccaccatgtggttgctgggatttgaactcaggaccttcagaagagcagtcagtgctcttacccgctgagccatctcgccgggtaatgtctaccaacactcggtggtacaccaacgaccctaaacttgagtcctggaagtcttctcgtcagtcacgagaatgggcgactcggtagagcggaaccccagcctggcctttcttatgtttcatattctaaattcaaactctgtaagtaaattcttaacaataaagtgcattgtggaggactagggagacggctttggggtcggaccggaaagaatacaaagtataagatttaagtttgagacattcatttaagaattgttatttcacgtaacacctcctgatccctctgccgacagcaggtaagggtgttttctggtgaacctgaccgccatagcttgatccccaaaacccatttgatggaaggggagaactgttgactttctctctctctctgtcgtccattcccacaaaagaccacttggactggcggtatcgaactaggggttttgggtaaactaccttcccctcttgacaactgaaagagagagagagactctctctctctctctctctctctcaaaatacatcaggattgctcattaaattaaaaaatgtgtatgttgagcctgttaacatatttttttagatttattgagagagagagagagagagagagagttttatgtagtcctaacgagtaatttaattttttacacatacaactcggacaattgtataaaaaaatctaaataatatttttattttatgtgtatgggcatcttgcctgcatgtgtctgtacaatgtctatggaggccagaagagggcggagataaaaataaaatacacatacccgtagaacggacgtacacagacatgttacagatacctccggtcttctcccgcctc

(19) United States
(12) Patent Application Publication
(10) Pub. No.: US 2006/0123501 A1
(43) Pub. Date: Jun. 8, 2006

(54) Method for treating inflammatory disorders

(76) Inventors: Robert G. Korneluk, Ottawa (CA); Martin Holcik, Ottawa (CA); Damiano Conte, Ottawa (CA); Charles Lefebvre, Bourget (CA); Eric LaCasse, Ottawa (CA)

Correspondence Address: AEGERA THERAPEUTICS INC., 810 CHEMIN DU GOLF, VERDUN, QC, H3E-1A8, CA

(21) Appl. No.: 11/292,007

(22) Filed: Dec. 2, 2005

Related U.S. Application Data
(60) Provisional application No. 60/632,952, filed on Dec. 6, 2004.

(51) Int. Cl. A01K 67/027; A61K 38/20; C12N 5/06

(52) U.S. Cl. 800/14; 424/85.2; 435/325

(57) ABSTRACT

The invention features a method of treating an inflammatory disorder, such as sepsis, in a subject. The method involves administering to the subject an antagonist of cIAP2 expression and/or function, so that the disorder is treated.

### CROSS REFERENCE TO RELATED APPLICATIONS

Applicants hereby claim priority from previously filed U.S. provisional patent application No. 60/632,952, filed on Dec. 6^{th}, 2004, the entire contents of which are hereby incorporated by reference.

### FIELD OF THE INVENTION

The present invention concerns a method for treating inflammatory disorders, and more particularly a method of treating sepsis.

### BACKGROUND OF THE INVENTION

The activation of the nuclear factor-κB (NF-κB) family of transcription factors rapidly induces the upregulation of inflammatory and anti-apoptotic genes including the cellular inhibitor of apoptosis 2 (cIAP2, also known as HIAP1 or BIRC3) (25). The ciap2 gene was first identified as a member of the evolutionarily conserved inhibitor of apoptosis (IAP) family of proteins (14) that are critical repressors of apoptosis. In addition, cIAP2 is a highly inducible gene that, along with cIAP1, is a component of the TNF receptor 2 (TNFR2) complex and therefore is a constituent of the TNFα signaling pathway (19). cIAP2 has been demonstrated to inhibit cell death by directly binding repressing the pro-apoptotic activity of a family of cysteine proteases (25), caspases, as well as targeting pro-apoptotic components of the TNFα signaling pathway for ubiquitin degradation (17). Despite these findings, the precise anti-apoptotic mechanisms as well as a pathophysiological role for cIAP has yet to be determined.

The extent of cytokine response to inflammatory agents, such as lipopolysaccharide (LPS), the biologically active element of the bacterial Gram negative membrane component endotoxin, is regulated by NF-κB. Under normal conditions an inflammatory response is beneficial in controlling invading pathogens and in clearing debris. However, a systematic activation of host macrophages by LPS can induce a hyper-inflammatory response resulting in pathogenic endotoxic shock (23). LPS-induced activation of macrophages is typically associated with the production of inflammatory mediator cytokines such as tumor necrosis factor-α(TNFα) and interleukin-1β (11). These cytokines act synergistically in the initiation of the inflammatory cascade of sepsis (2) resulting in hypotension, tachycardia, systemic edema, disseminated intravascular coagulation and finally multiple organ failure.

LPS specifically binds the macrophage cell-surface receptor, CD14 (6), which subsequently interacts with the Toll-like receptor 4 (TLR4) (1). TLR4 next recruits the toll-adaptor protein, myeloid differentiation factor 88 (MyD88) (26), to activate NF-κB and thereby induce the upregulation of pro-inflammatory cytokines. Activation by LPS of a macrophage results in enhanced phagocytosis of bacteria and the release of cytokines prompting other macrophages, phagocytes and T cells to the site of infection. This initiates a pro-inflammatory response and thereby influences the nature of the adaptive immune response. Macrophages are now well recognized to be the primary mediators for the lethal effects caused by bacterial- or LPS-induced septic shock (10).

LPS activation is known to impart a macrophage with an increased resistance against apoptotic triggers. An inflammatory response produces nitric oxide (NO), reactive oxygen intermediates (ROI) and the upregulation of Fas ligand on immune-regulating lymphocytes, all of which are detrimental to both invading pathogens and resident cells. This LPS-induced apoptotic resistance is essential for macrophages to function within an inherently hostile, anti-microbial pro-inflammatory environment. Considerable interest in the function of cIAP2 has arisen from its role as a major NF-κB-regulated survival factor. cIAP2 has been suggested to be the essential component that is chiefly responsible for protecting rat hepatocytes from LPS-induced lethal assault (22).

Given that cIAP2 is a potential key survival factor induced via NF-κB activation in many cells, including macrophages, it would be important to determine whether cIAP2 is an essential component during an innate pro-inflammatory response. Generating transgenic mice, which include mutations in the genes of interest, so called “knockout” mice, would be an ideal research tool for this. Thus far, no ciap2 knockout mice exist and therefore there exists a need to develop a transgenic non-human mammal in which the ciap2 gene has been modified. Using these “knockout” mice to determine the role of cIAP during the aforesaid pro-inflammatory response would be valuable in developing therapeutics to treat inflammatory disorders in humans.

### SUMMARY OF THE INVENTION

The cellular inhibitor of apoptosis 2 (cIAP2/HIAP1) is a potent inhibitor of apoptotic death. In contrast to other members of the inhibitor of apoptosis (IAP) family, cIAP2 is transcriptionally inducible by NF-kB in response to multiple apoptotic triggers. We have discovered that cIAP2 knockout mice, cIAP2^{-/-}, exhibit profound resistance to lipopolysaccharide (LPS)-induced sepsis specifically because of an attenuated inflammatory response. We have shown that LPS potently up-regulates cIAP2 in macrophages and that cIAP2^{-/-} macrophages are highly susceptible to apoptosis in a LPS-induced pro-inflammatory environment, thereby demonstrating that cIAP2 maintains a normal innate immune inflammatory response. Advantageously, this discovery therefore provides a new way in which to treat sepsis and other inflammatory disorders in humans by inhibiting cIAP2 protein expression and/or function in cells such as macrophages and causing them to undergo apoptosis, thereby significantly reducing or essentially eliminating the inflammatory cascade in sepsis.

In accordance with an embodiment of the present invention, there is provided a method of treating an inflammatory disorder in a subject, the method comprising: administering to the subject in need thereof an antagonist of cIAP2 expression and/or function, thereby treating the disorder. In one example, the antagonist antagonizes cIAP2 protein function and the inflammatory disorder is characterized by cells which produce cytokines. In one example, the cells comprise macrophages, T-cells, or fibroblasts and the cytokines are IL-1β or TNF-α.

In accordance with another embodiment of the present invention, there is provided a method of causing apoptosis in cells, the cells being characterized by producing cytokines, the method comprising: antagonizing cIAP2 expression and/or function by contacting the cells with a cIAP2 antagonist, thereby causing the cells to undergo apoptosis. In one example, the antagonist antagonizes cIAP2 protein function and the cells are from a subject suffering from an inflammatory disorder, the disorder being characterized by cells which produce cytokines. In one example, the cells comprise macrophages, T-cells, or fibroblasts. In another example, the cytokines produced by the cells are IL-1β or TNF-α.

In accordance with one aspect of the present invention, there is provided a method of treating sepsis in a subject, the method comprising: administering to the subject in need thereof an antagonist of cIAP2 expression and/or function, thereby treating the sepsis.

In accordance with another embodiment of the present invention, there is provided a disrupted ciap2 gene which comprises a nucleic acid sequence, according to SEQ ID NO. 1.

In accordance with one aspect of the present invention, there is provided a disrupted ciap2 gene which consists of a nucleic acid sequence, according to SEQ ID NO.1.

In accordance with another embodiment of the present invention, there is provided a transgenic non-human mammal comprising the disrupted ciap2 gene, as described above. In one example, the transgenic non-human mammal is a mouse.

In accordance with one aspect of the present invention, there is provided a transgenic non-human mammal model for studying sepsis or septic shock, wherein the mammal comprises the disrupted ciap2 gene, as described above.

In accordance with yet another aspect of the present invention, there is provided a transgenic non-human mammal model for studying infection, wherein the mammal comprises the disrupted ciap2 gene, as described above.

In accordance with still another aspect of the present invention, there is provided a transgenic non-human mammal whose genome is heterozygous for a disruption in the ciap2 gene, as described above, wherein the disruption in a homozygous state inhibits the production of function cIAP2 protein, which results in a transgenic non-human mammal having a reduced severity of sepsis as compared to a wild type mammal.

In accordance with yet another aspect of the present invention, there is provided a transgenic non-human mammal whose genome is homozygous for the disrupted ciap2 gene, as described above, the disrupted gene in a homozygous state inhibiting the production of functional cIAP2 protein, which results in a transgenic non-human mammal having a reduced severity of sepsis as compared to a wild type mammal.

In accordance with another embodiment of the present invention, there is provided a cell which is isolated from the transgenic non-human mammal, in which the genome of the cell comprises the homozygous disrupted ciap2 gene, as described above, wherein the disruption of the ciap2 gene inhibits production of functional cIAP2 protein.

In accordance with another embodiment of the present invention, there is provided a primordial germ cell which is isolated from a transgenic non-human mammal embryo whose genome comprises a homozygous disrupted ciap2 gene, as described above, wherein the disruption of the ciap2 gene inhibits production of functional cIAP2 protein.

In accordance with another embodiment of the present invention, there is provided a cell line comprising a progeny of the cell, as described above, wherein the progeny of the cell comprise a homozygous disrupted ciap2 gene, wherein the disruption inhibits production of functional cIAP2 protein.

In accordance with another embodiment of the present invention, there is provided a method for producing a heterozygous non-human mammal, the mammal having somatic and germ cells containing a gene coding for a disrupted mammal cIAP2 protein, as described above, the method comprising:
(a) introducing the gene into an embryo blastocyst;
(b) transplanting the embryo into a pseudopregnant mammal, the blastocyst developing to term;
(c) identifying a mammal which carries a disruption in one allele, the mammal exhibiting a reduced severity of sepsis as compared to a wild type mammal.

In accordance with another embodiment of the present invention, there is provided a method for producing a homozygous non-human mammal having somatic and germ cells which contain a gene encoding a disrupted mammalian cIAP2 protein, as described above, the method comprising:
(a) introducing the gene into an embryo blastocyst;
(b) transplanting the embryo into a pseudopregnant non-human mammal the blastocyst developing to term;
(c) identifying a first non-human mammal which carries the disrupted gene;
(d) inter-breeding the first mammal with a second non-human mammal carrying the disrupted gene; and
(e) identifying a non-human mammal which carries the disrupted gene in both alleles, the non-human mammal exhibiting a pathophysiological phenotype which is characterized by a substantial absence of expression of non human mammal cIAP2 protein, and by a reduced severity of sepsis.

In accordance with another embodiment of the present invention, there is provided a method of testing the transgenic non-human mammal, as described above, for the severity of septic shock or endotoxic shock, the method comprising challenging the mammal with the shock and evaluating the effect of the severity of septic shock or endotoxic shock.

In accordance with another embodiment of the present invention, there is provided a vector comprising in the 5′ to 3′ direction a 5′ arm homologous to the cIAP2 gene; a marker sequence; and a 3′ arm homologous to the cIAP2 gene. In one example, the marker sequence comprises a splice acceptor site, a bicistronic gene encoding beta-galactosidase, and an IRES-driven beta-galactosidase-neo fusion protein expression gene. In another example, the IRES-driven expression gene replaces exons 2 to 5 of cIAP2.

In accordance with one aspect of the present invention, there is provided a cell comprising the vector, as described above. In one example, the cell is a mouse embryonic stem cell.

In accordance with another aspect of the present invention, there is provided a method for inducing apoptosis in a mammalian cell, the method comprising: administering directly to the cell the disrupted ciap2 gene, as described above, so as to disrupt cIAP2 protein expression or function in the cell.

In accordance with one embodiment of the present invention, there is provided a method of screening compounds for treating sepsis or septic shock, the method comprising:
a) applying a sepsis or septic shock challenge to the transgenic non-human mammal, as described above, the mammal exhibiting resistance to septic shock;
c) administering a test compound to the mammal;
d) determining the effect of the test compound on the severity of sepsis or septic shock in the mammal; and
e) correlating the effect of the test compound on septic shock or sepsis of the mammal with an effect of the test compound on the macrophages in a non treated mammal having a disrupted ciap2 gene, or in a wild type mammal.

In accordance with another aspect of the present invention, there is provided a method of reducing mortality of sepsis, septic shock, endotoxic shock in a wild type mammal, the method comprising: comprising administering to the mammal in need thereof an antagonist of cIAP2 protein function and/or expression, thereby reducing the mortality of sepsis, septic shock, endotoxic shock

In accordance with another aspect of the invention, there is provided a method for reducing mortality of trauma in a wild type mammal, the method comprising: comprising administering to the mammal in need thereof an antagonist of cIAP2 protein function and/or expression, thereby reducing the mortality of trauma.

In accordance with another aspect of the present invention, there is provided a plurality of cells derived from a transgenic non human animal, the cells comprising the disrupted ciap2 gene, as described above.

In accordance with another embodiment, there is provided a kit for determining the sensitivity of macrophages to apoptosis stimuli, the kit comprising a vial for receiving a sample of macrophages from the transgenic mammal, as described above, after a septic shock or sepsis challenge; a stain for staining the macrophages for Annexin V; and instructions for comparing the stained macrophages with a control mammal.

In accordance with another aspect of the present invention, there is provided a method for treating sepsis in a subject, the method comprising: providing in the subject in need thereof one or more cells, the cells being capable of producing progeny cells having disrupted cIAP2 protein expression and/or function, wherein the cells express the disrupted ciap2 gene, as described above, under the control of a constitutive, inducible, or cell specific promoter so as tot cause apoptosis of the cells relative to untreated control cells not expressing cIAP2, thereby treating the sepsis.

Examples of inflammatory disorders are selected from inflammatory peritonitis, osteoarthritis, acute pancreatitis, chronic pancreatitis, asthma, adult respiratory distress syndrome, glomerulonephritis, rheumatoid arthritis, systemic lupus erythematosus, scleroderma, chronic thyroiditis, Graves' disease, autoimmune gastritis, insulin-dependent diabetes mellitus (Type I), autoimmune hemolytic anemia, autoimmune neutropenia, thrombocytopenia, chronic active hepatitis, myasthenia gravis, inflammatory bowel disease, Crohn's disease, psoriasis, atopic dermatitis, graft vs. host disease, osteoporosis, multiple myeloma-related bone disorder, leukemias and related disorders, myelodysplastic syndrome, acute myelogenous leukemia, chronic myelogenous leukemia, metastatic melanoma, Kaposi's sarcoma, multiple myeloma, sepsis, septic shock, Shigellosis, Alzheimer's disease, Parkinson's disease, cerebral ischemia, myocardial ischemia, spinal muscular atrophy, multiple sclerosis, AIDS-related encephalitis, HIV-related encephalitis, aging, alopecia, neurological damage due to stroke, ulcerative collitis, infectious hepatitis, juvenile diabetes, lichenplanus, acute dermatomyositis, eczema, primary cirrhosis, uveitis, Behcet's disease, atopic skin disease, pure red cell aplasia, aplastic anemia, amyotrophic lateral sclerosis, nephrotic syndrome, burns, bronchitis, tendinitis, bursitis, periarteritis nodosa, thyroiditis, Hodgkin's disease, rheumatic fever, sarcoidosis, polymyositis, gingivitis, hypersensitivity, conjunctivitis, swelling occurring after injury, allergic rhinitis, endotoxin shock syndrome, and atherosclerosis, psoriatic arthritis, vasculitis, Polymyalgia, Rheumatica, Wegener's granulomatosis, temporal arteritis, chronic obstructive pulmonary disease, cryoglobulinemia, transplant rejection and ataxia telangiectasia. In one example, the inflammatory disorder is sepsis. In one example, the inflammatory disorder is LPS or IL-7 induced. In another example, the subject is a human.

### BRIEF DESCRIPTION OF THE DRAWINGS

Further aspects and advantages of the present invention will become better understood with reference to the description in association with the following Figures, wherein:

FIG. 1. The disruption of ciap2 gene by homologous recombination.

The structure of the 5′ end of the mouse ciap2 locus, targeting vector and the targeted ciap2 allele are shown (A). The opened black box shows the position of the probe used for the genomic Southern blot analysis. Southern blot analysis of Eco-RV-digested genomic DNA of embryonic stem cells (B) reveals the presence of the targeted (15 kb) and wild type (27 kb) alleles. Western blot analysis of lung protein extracts indicates absence of the 66 kDa cIAP2 full length polypeptide XIAP and surviving protein levels are also shown (C). The relative quantitative levels of cIAP2, cIAP1 and xiap mRNA, which was derived from mouse embryonic fibroblast of cIAP2^{-/-} and wild-type littermate mice are shown in (D). Results are mean±s.d. (n=6, average of triplicate wells per mouse, P<0.05).

FIG. 2. cIAP2^{-/-} mice are resistant to LPS.

The iap mRNA levels of LPS-treated peritoneal macrophages derived from wild type C57Bl/6 mice relative to untreated controls were assayed. (A) xiap, ciap1, and ciap2 message levels of macrophages exposed to a range of LPS doses relative to untreated controls. (B) ciap2 mRNA message of macrophages exposed to LPS was assayed over 24 h. Results are mean±s.d. (n=5, average of triplicate wells per mouse, P<0.05).

FIG. 3. cIAP2^{-/-} mice are resistant to LPS-induced endotoxic shock.

Mice were injected IP with a range of indicated LPS doses (n=15). (A) cIAP2^{-/-} mice (solid shapes), all cIAP2^{-/-} mice survived for up to 7 days at an LPS dose of 40 mg/kg and below) and littermate control (open shapes) were treated with a range of LPS doses. (B) cIAP2^{-/-} mice (solid shapes) and littermate controls (open shapes, all littermate controls mice did not survive past the first day at an LPS dose of 60 mg/kg and above) were given a higher range of LPS doses (P<0.001).

FIG. 4. Macrophage cell counts and function is not impaired in cIAP2^{-/-} mice.

Spleen- and peritoneal-derived macrophage cell count numbers are shown (A) for cIAP2^{-/-}, cIAP2^{+/-} and cIAP2^{+/+} mice (n=10). Both cIAP2^{-/-} and littermate control-derived macrophages stained for the LPS binding surface receptor CD14 (B). The proliferation of B cells for cIAP2^{-/-} mice and littermate controls of cIAP2^{+/+} after culturing with the indicated range of LPS doses (n=6) (C). Cultured macrophages from cIAP2^{-/-} or littermate control cIAP2^{+/+} mice were exposed to LPS for 10 h and subsequently IL-1β (D) or TNF-α (E) levels were measured by ELISA (n=6). Additionally, macrophages were exposed to the indicated range of LPS doses for 24 h and IL-1β (F) or TNF-α (G) levels were determined (n=6). Results are mean±s.d. in triplicate per mouse, P<0.01

FIG. 5. cIAP2^{-/-}-derived macrophages display an increased sensitivity to apoptosis.

Peritoneal-derived macrophages from cIAP2^{-/-} (lower panels) and cIAP2^{+/+} mice (upper panels) were either pre-treated with LPS for 4 h or not pre-treated prior to exposure to α-Fas antibody and then TUNEL stained to assess cell viability (A). Percentages of viable cells (n=5, average of triplicate wells per mouse, P values were <0.01) are shown within each TUNEL stained panel along with the standard deviation. cIAP2^{-/-}-derived (open shapes) and cIAP2^{+/+}-derived (solid shapes) T cells were pre-incubated with a range of IL-7 concentrations and then exposed to dexamethasone and T cell survival was monitored over a 12 h period (B) (n=3, average of triplicate wells per mouse, results are mean±s.d., P values at the 12 h point were <0.05). Peritoneal-derived cell numbers are shown for macrophages of cIAP2^{-/-} mice and littermate control cIAP2^{+/+} mice injected i.p. with LPS(C). The combined number of T and B cells of cIAP2^{-/-} and littermate control mice that had been i.p. injected with LPS are shown (D).

FIG. 6. Reduced liver-derived macrophage cell counts in cIAP2^{-/-} mice treated with LPS.

Liver tissue derived from cIAP2^{-/-} (upper panels) and cIAP2^{+/+} mice (lower panels) pre-treated with LPS (35 mg/kg) for 2 hours (a) or 6 hours (b). cell counts (n=5, average of 3 sections (average of 5 fields per section) per mouse, P<0.01) are shown within each anti-F4/80 FITC conjugated antibody stained panel along with the standard deviation.

### DETAILED DESCRIPTION OF THE INVENTION

Definitions

Unless otherwise specified the following definitions apply:

The singular forms “a”, “an” and “the” include corresponding plural references unless the context clearly dictates otherwise.

As used herein, the term “comprising” or “comprises” is intended to mean that the list of elements following the word “comprising” are required or mandatory but that other elements are optional and may or may not be present.

As used herein, the term “consisting of” or “consists of” is intended to mean including and limited to whatever follows the phrase “consisting of”. Thus the phrase “consisting of” indicates that the listed elements are required or mandatory and that no other elements may be present.

As used herein, the term “disrupted gene” is intended to refer to a gene which contains an insertion, substitution or deletion resulting in the loss of substantially all of the biological activity associated with the gene. For example, a disrupted ciap2 gene would be unable to express a substantial amount of cIAP2 protein. The disrupted ciap2 gene of the present invention comprises a nucleic acid sequence, according to SEQ ID NO: 1.

As used herein, the term “endotoxic shock” or “septic shock” is intended to mean shock that is induced by bacterial endotoxins, such as produced by E. coli, staphyloccous, streptococcus, meningococcus and the like. Such bacterial endotoxins include, but are not limited to, LPS and the like.

As used herein, the term “sepsis” is intended to mean the presence of various pus forming, and other pathogenic organisms or their toxins, in the blood or tissues of the mammal. One common form of sepsis is Septicemia.

As used herein, the term “transgenic non human mammal” is intended to refer to an organism, which contains a defined change to it's germline wherein the change is not ordinarily found in the Wild Type organism. The change may be passed on to the organism's progeny. The change in the organism's germline may be an insertion, a substitution or a deletion. The insertion, deletion or substitution may result in the elimination of a phenotype associated with the disrupted gene. The term “transgenic” further encompasses organisms, which contain modifications to their existing genes and organisms which are modified to contain exogenous genes introduced into their germline. Examples of “transgenic non-human mammals” includes animals such as mice, rats, guinea pigs, rabbits, dogs, sheep, swine, cows, goats and horses, and non-human primates, such as monkeys and chimpanzees.

As used herein, the term “Wild Type”, when referring to a mammal, is intended to mean a mammal having a normal allele or phenotype.

As used herein, the term “allele” is intended to mean an alternative form of a gene that may occur at a given gene locus.

As used herein, the term “targeting vector” is intended to mean a vector which comprises nucleic acid sequences (transgenic DNA) which can be inserted into a gene to be disrupted for example by homologous recombination, or by RNA interference. In the Examples of the present invention, the targeting vector does not include a promoter. It uses the endogenous cIAP2 promoter after correct integration of the targeting vector. In the direction 5′-3′ the vector contains a 5′ arm homologous to cIAP2 and then a ‘marker’ or selection sequence (of 7 kb) in the middle, which includes a splice acceptor site and a bicistronic gene encoding beta-galactosidase and an Internal Ribosomal Entry Site (IRES) (from EMCV virus)) driver of expression of beta galactosidase-neo fusion protein, (we designate this insert or cassette as “SA-Beta geo”), and finally a 3′ arm homologous to cIAP2. It is to be understood that the marker sequence may include several markers known to those skilled in the art. The genomic locus includes the SA-Beta geo marker sequence of 7 kb, which is found between the 2 recombination arms (5′ and 3′ that are homologous to cIAP2). The disruption of the cIAP2 sequence occurs by homologous recombination which will, in some cases, ‘loop’ out 8 kb encoding the ATG and first 6 exons of cIAP2. These positive recombinants can be screened for by neo selection and southern analysis, or PCR analysis. Thus the two recombination arms allow for pairing of the targeting vector with the genomic sequence and when integrated properly, the SA-beta gal-IRES-neo cassette is inserted in place of the 8 kb cIAP2 sequence (the disrupted sequence spanning the ATG and the first 6 exons). Thus this provides a mouse with a cIAP2 promoter driving expression of beta galactosidase-neo fusion protein instead of normal cIAP2 production. The heterozygous mice are crossed to generate homozygous mice to disrupt both alleles of cIAP2.

As used herein, the term “transgenic DNA” is intended to mean the polynucleotide comprised within the transgene, which polynucleotide encodes the protein of interest. The present invention relies on the use of a nucleic acid construct to generate a transgenic non-human mammal.

As used herein the terms “apoptosis” is intended to mean the process of cell death in which a dying cell displays a set of well-characterized biochemical indicia that include cell membrane blebbing, cell soma shrinkage, chromatin condensation, and DNA fragmentation.

As used herein, the term “apoptosis stimuli” or “apoptosis trigger” are used interchangeably and are intended to mean activators of a cell death receptor such as Fas ligand, TRAIL, TNF-α, TNF-β, and the like.

As used herein, the term “subject” or “patient” is used interchangeably and is intended to mean mammals such as humans, primates, rats, mice, guinea pigs, goats, sheep, horses, pigs and the like.

As used herein, the term “treating sepsis” is intended to mean prophylactic treatment of subjects, including humans, who are suffering from sepsis so that the symptomology is reduced or essentially eliminated.

As used herein, the term “reduced severity of sepsis” is intended to mean a reduction in symptomology and a reduced likelihood of sepsis induced death or multiple organ failure.

As used herein, the term “treating an inflammatory disorder” is intended to mean that an effective amount of the cIAP2 antagonist is given to the subject in an amount sufficient to effect beneficial or desired clinical results. This can be administered in one or more administrations. The amount administered is sufficient to palliate, ameliorate, stabilize, reverse, slow or delay progression of an inflammation-related condition in accordance with clinically acceptable standards for disorders to be treated. Detection and measurement of indicators of efficacy may be measured by a number of available diagnostic tools, including but not limited to, for example, by physical examination including blood tests, pulmonary function tests, and chest X-rays; CT scan; bronchoscopy; bronchoalveolar lavage; lung biopsy and CT scan.

As used herein, the term “inflammatory disorder” is intended to mean a disease or disorder characterized by, caused by, resulting from, or becoming affected by inflammation. An inflammatory disorder may be caused by or be associated with biological and pathological processes associated with NF-κB mediated processes.

As used herein, the term “ciap2 gene” is intended to mean a gene which encodes a polypeptide having at least one BIR domain and which is capable of modulating (inhibiting or enhancing) apoptosis in a cell or a tissue when provided by intracellular or extracellular delivery methods (see for example U.S. Pat. No. 5,919,912). The ciap2 gene is a gene having a nucleotide sequence that is described in U.S. Pat. No. 6,156,535, which is hereby incorporated by reference.

As used herein, the term “cIAP2” protein or “cIAP2 polypeptide” is intended to mean a polypeptide, or a fragment thereof, which is encoded by the ciap2 gene. The sequence of the cIAP2 protein is disclosed in U.S. Pat. No. 6,156,545, which is hereby incorporated by reference. It is to be noted that according to convention the italicized “ciap2” refers to the gene, whereas the non-italicized “cIAP2” refers to the protein.

As used herein, the term “cell” is intended to mean a single cellular organism, a cell from a multi cellular organism or a cell contained within a multi cellular organism. Examples of cells include, but are not limited to, macrophages, T lymphocytes, B cells, and fibroblasts.

As used herein, the term “transgene” is intended to mean any piece of DNA, which is inserted by artifice into a cell and typically becomes part of the genome of the organism that develops from that cell. Such a transgene may include a gene that is partly or entirely heterologous (i.e. foreign) to the transgenic organism or may represent a gene which is homologous to an endogenous gene of the organism, or may be a short-hairpin RNA that targets and degrades cIAP mRNA by RNA interference.

As used herein, the term “IAP gene” is intended to mean a gene encoding a polypeptide having at least one BIR domain and which is capable of modulating (inhibiting or enhancing) apoptosis in a cell or tissue. The IAP gene is a gene having about 50% or greater nucleotide sequence identity to at least one of NAIP (Birc 1), HIAP-1 (cIAP2, API2, MIHC, hITA), HIAP-2 (cIAP1, HIHB), XIAP (hILP, hILP1, MIHA, API3), survivin (TIAP, MIHD, API4), livin (KIAP, ML-IAP, cIAP3, HIAP3), and BRUCE. The region of sequence over which identity is measured is a region encoding at least one BIR domain and a ring zinc finger domain. Mammalian IAP genes include nucleotide sequences isolated from any mammalian source. The mammal is typically a human.

As used herein, the term “protein”, “polypeptide” or “polypeptide fragment” is intended to mean any chain of more than two amino acids, regardless of post-translational modification, for example, glycosylation or phosphorylation, constituting all or part of a naturally occurring polypeptide or peptide, or constituting a non-naturally occurring polypeptide or peptide.

As used herein, the term “IAP protein” or “IAP polypeptide” is intended to mean a polypeptide or protein, or fragment thereof, encoded by an IAP gene. Examples of IAP polypeptides include, but are not limited to NAIP (Birc 1), HIAP-1 (cIAP2, API2, MIHC, hITA), HIAP-2 (cIAP1, HIHB), XIAP (hILP, hILP1, MIHA, API3), survivin (TIAP, MIHD, API4), livin (KIAP, ML-IAP, cIAP3, HIAP3), and BRUCE.

As used herein, the term “antagonist of cIAP2 expression and/or function” is intended to mean any activity, which inhibits or decreases in vivo or in vitro, the expression of the ciap2 gene as compared to non-antagonized ciap2 gene. The expression of the cIAP2 protein may be completely or partially suppressed, or the function of the cIAP2 protein maybe completely or partially suppressed.

In the methods where a “test compound” is contacted with a cell of the invention, the method may be intended to identify antagonists of cIAP2 expression and/or function. Reversal or prevention of the effect of cIAP2 function in a cell of the invention by a test compound is an indication that the test compound may be an antagonist of cIAP2 expression and/or function. Suitable test compounds which may be tested in the above methods include antibody products (for example, monoclonal and polyclonal antibodies, single chain antibodies, chimeric antibodies and CDR grafted antibodies). Furthermore, combinatorial libraries, defined chemical identities, small molecules, peptide and peptidomimetics, oligonucleotides and natural product libraries, such as display libraries (e.g. phage display libraries) may also be tested. The compounds may be chemical compounds. Batches of the candidate substances may be used in an initial screen of, for example, ten substances per reaction, and the substances of batches which show inhibition may be tested individually.

I. Transgenic Non-Human Mammals

Transgenic non human mammals according to the present invention which comprise a disrupted ciap2 gene (mutation) in all of the diploid cells, may be obtained using methods well known in the art. Generally speaking, the mutation may be introduced into target cells for example ES cells by injection or transfection of a targeting DNA vector. The vector or construct contains a genomic segment of the cIAP2 gene with the desired mutation in place. (see “Manipulating the Mouse Embryo” in Hogan et al., eds., 2ed., Cold Spring Harbor Press, 1994). One route for introducing foreign DNA into a germline involves the direct micro injection of linear DNA molecules into a oocyte which are then implanted into pseudo pregnant foster females. Alternatively, the transfected ES cells may be injected into appropriate blastocysts which are then implanted into foster females. “Knockout Mice” are a specific type of transgenic mammal which are obtained by making mutant ES cells. The ES cells are injected into blastocysts to produce chimeric mice (heterozygotes) and then the chimeras are bred to obtain homozygotes to obtain the germline transmitted mutation.

The transgenic non-human mammals, according to the present invention, may serve as animal models for studying the pathology of sepsis or septic shock. Therapeutic compounds, useful in treating one of these conditions, may be screened using the transgenic non-human mammals or cell-lines derived there from.

The targeting vector includes a plasmid backbone, pKO, which contains two pieces of cIAP2 genomic sequence and Bgeo (fusion of beta-galactocidase and neomycin) cassette which is used as a selection marker. The Bgeo is a promoterless cassette, which means that it is driven by endogenous cIAP2 promoter. As a result lacZ may be used to follow the expression of cIAP2. This may be used for screening small molecules that regulate the cIAP2 promoter. Moreover, this may be used to screen for NF-kB antagonists since cIAP2 is an NF-kb regulated gene.

Establishment of cIAP2^{-/-} Mice.

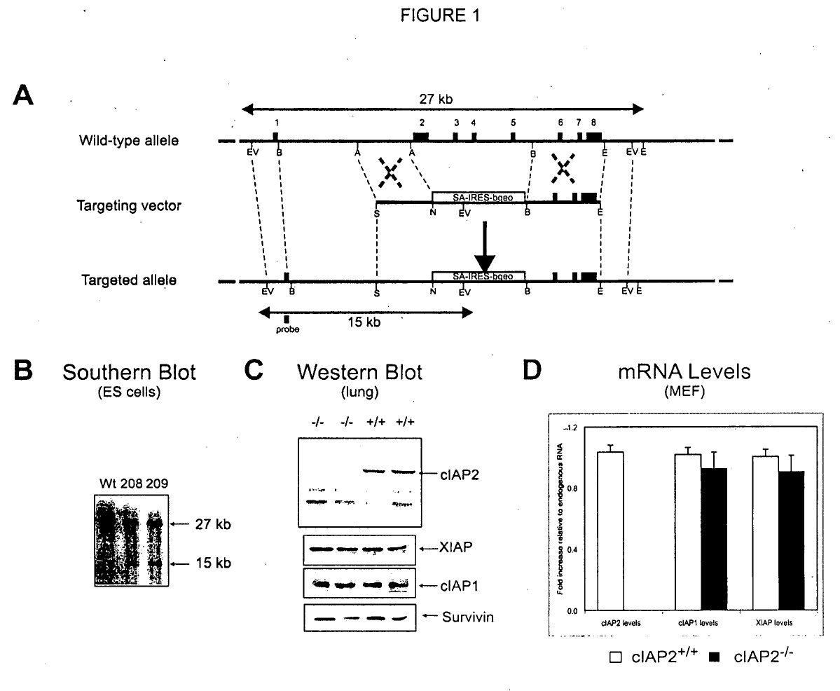

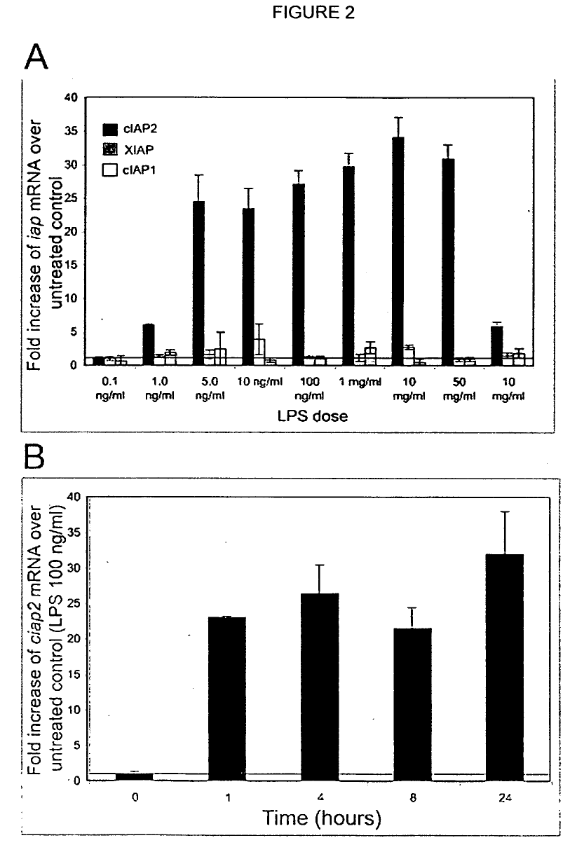

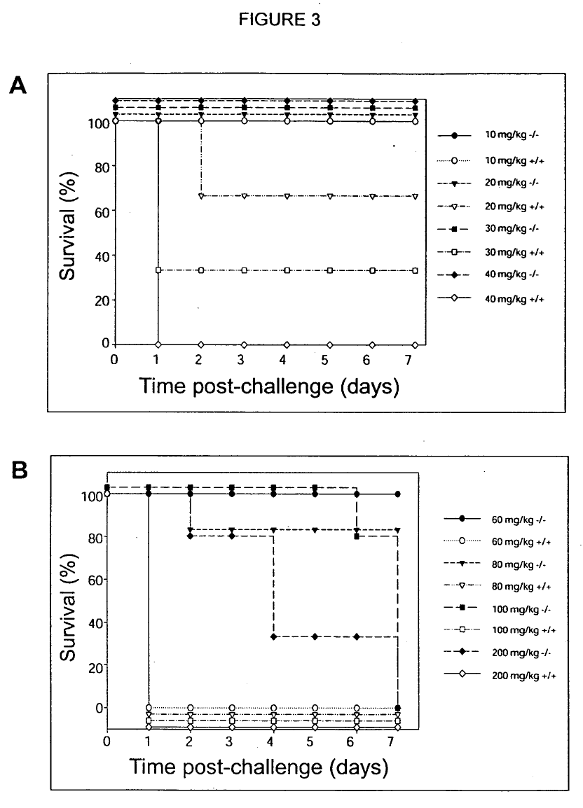

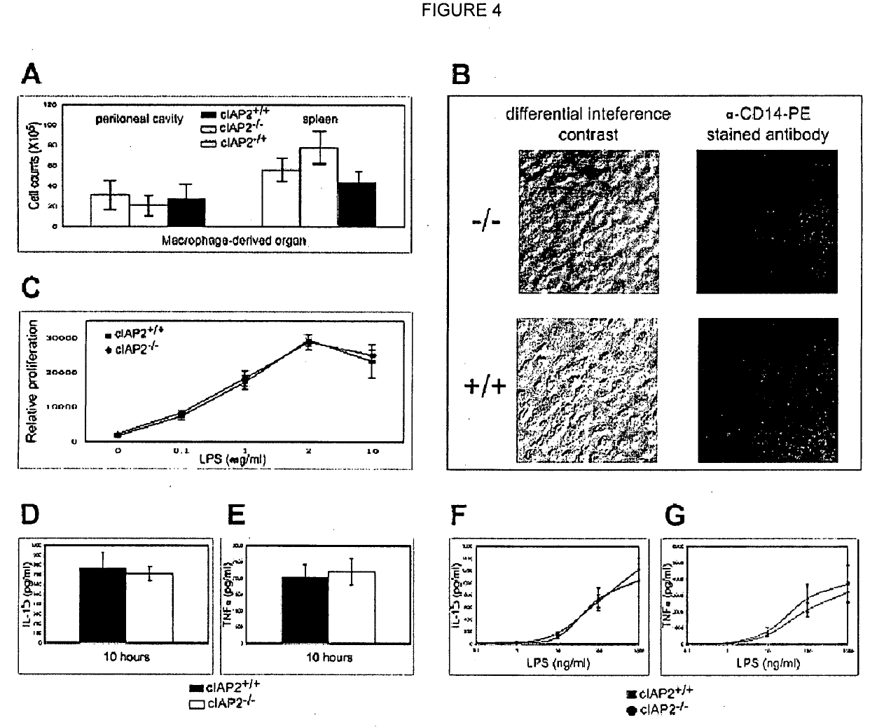

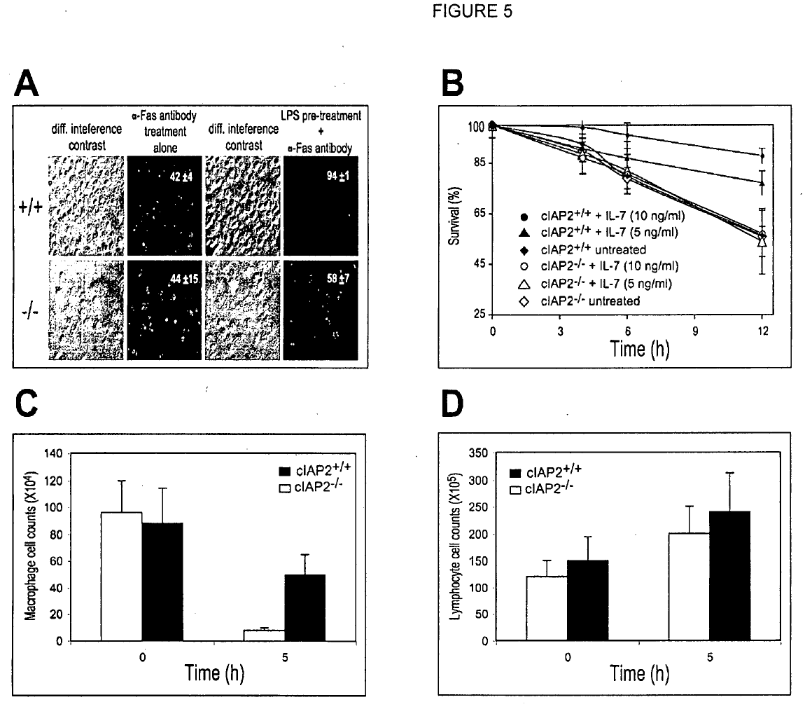

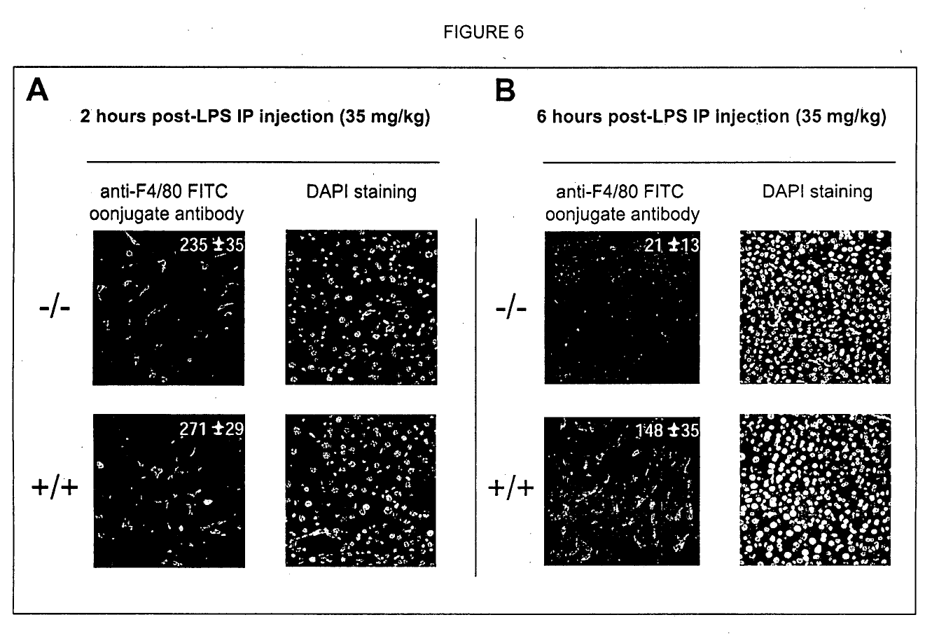

To study the function of cIAP2, the murine ciap2 gene was disrupted by homologous recombination in embryonic stem cells (FIGS. 1A and 1B). cIAP2^{-/-} mice showed no overt phenotype, were fertile, followed Mendelian frequency of inheritance and appeared healthy up to 52 weeks of age. Whole mouse and organ weights as well as primary and secondary lymphoid compartment cell count numbers for cIAP2^{-/-} and control littermates, up to the age of 36 weeks, showed no significant difference (data not shown). Western blot analysis and quantitative RT-PCR showed no compensatory increase of IAP family members in cIAP2 ablated tissue (FIGS. 1C and D). Mutant mice having the disrupted ciap2 gene may also be generated using gene targeting techniques.

cIAP2 is Highly Upregulated in Macrophages Treated with LPS.

LPS-induced activation of macrophages causes the upregulation of a multitude of genes, including the production/release of inflammatory mediators, the upregulation of cell surface receptors and of cell survival proteins. Conflicting previous reports suggested that in macrophages cIAP2 is upregulated, or remains unchanged, in response to LPS treatment. As well, it has been previously suggested that either XIAP or cIAP1 levels would rise to compensate for the loss of cIAP2. Therefore, the iap mRNA levels of LPS-treated macrophages, derived from the peritoneal cavity, were assayed relative to untreated controls. Peritoneal macrophages were cultured at 10⁵ cell/well (96 well, flat bottom plate) and exposed to varying concentrations of LPS for 18 hours. Xiap and ciap1 message levels remained relatively unchanged over the range of LPS doses (0.1 ng/ml to 10 mg/ml) (FIG. 2A). In contrast, ciap2 levels increased dramatically, up to 30 fold above untreated controls (P<0.01 for all LPS doses) (FIG. 2A). The innate immune system provides protection within the first minutes to hours after a pathogenic challenge. Therefore, a LPS-ciap2 mRNA time course assay was performed (100 ng/1 ml LPS). ciap2 mRNA message was substantially upregulated, >20 fold above untreated controls, within 1 hour and this increased level was maintained for 24 hours (FIG. 2B).

cIAP2′ Mice are Resistant to LPS-Induced Endotoxic Shock.

Schoemaker et al. proposed an important role of cIAP2 in LPS-induced death of liver cells sensitized to endotoxic shock by D-galactosamine (DGLN) (22). This work also predicted that cIAP2 null mice would therefore be highly susceptible to LPS-induced endotoxic shock. Thus to investigate the role of cIAP2 in an innate immune response, the cIAP2^{-/-} mice were treated with LPS. Surprisingly, contray to the predicted outcome, an intraperitoneal (IP) injected administration of LPS proved fatal to both wild-type (FIG. 3A) and cIAP2^{-/+} mice (data not shown), but not to cIAP2 null mice (FIG. 3A). All cIAP2^{-/-} mice treated with 40 mg LPS/kg or less survived, while littermate controls succumb in a dose dependent manner. In fact, the LD₁₀₀ cIAP2^{-/-} mice was found to be approximately 3× greater (100 mg LPS/kg) compared to that of control littermates (35 mg LPS/kg). Moreover, cIAP2^{-/-} mice survived 2-7 days even at a LPS dose of 200 mg LPS/kg, in contrast to control littermates that all died within 24 h, even at the lower dose of 40 mg LPS/kg (FIG. 3B).

cIAP2^{-/-} Mice are Susceptible to Fas-, Platelet-Activating Factor (PAF)- and D-Galactosamine/LPS-Induced Death.

In order to demonstrate the sensitivity of cIAP2^{-/-} mice to other lethal insults and inflammatory mediators, additional triggers were tested. The response of these animals to an IP injection of α-Fas antibodies (100 μg/mouse) (Table 1) and the effect of treatment with platelet-activating factor (PAF), an inflammatory mediator that acts downstream to the LPS activation of macrophages (24) (Table 2) was examined. In addition, we exposed cIAP2^{-/-} mice to a second mode of LPS-induced toxicity, where treatment with D-galactosamine (DGLBN) sensitized mice to endotoxic shock. In contrast to LPS alone, LPS with DGLN caused rapid demise of the mice (<3 hours, Table 3). In all cases, cIAP2^{-/-} mice and control littermates demonstrated similar sensitivity and died at identical rates.

cIAP2^{-/-} Mice Display an Attenuated Inflammatory Response.

LPS directly activates macrophages to produce large amounts of IL-1β and TNF-α and to mediate a cascade of events leading to endotoxic shock. We therefore assayed the levels of these pro-inflammatory cytokines in serum from cIAP2^{-/-} mice treated with LPS (35 mg LPS/kg). In cIAP2^{-/-} mice, IL-1β serum levels peaked at 4 h and then markedly dropped off (Table 4). This was in contrast to littermate controls where IL-1β levels decreased much later. Likewise, comparable initial TNF-α serum levels; however, the TNF-α levels dropped off to approximately 10 pg/ml in cIAP2^{-/-} mice by 10 hours, while in littermate controls TNF-α serum levels stabilized to approximately 400 pg/ml and were maintained until death (Table 4). As a further study, we also investigated the serum concentrations of IL-12 (Table 4) after an injection of LPS (35 mg/kg), yet again, we observed an attenuation of a macrophage cytokine, IL-12, by the 6 hour time point. Nevertheless, cIAP2^{-/-} mice did display early outward signs of sepsis, such as eye exudates and ruffled fur; however, their condition quickly ameliorated, corresponding to the observed waning of the LPS-induced inflammatory cytokines seen within the cIAP2^{-/-} mice.

Macrophage Cytokine Production and Cell Counts are not Impaired in cIAP2^{-/-} Mice

The inability of the cIAP2^{-/-} mice to sustain IL-1β and TNF-α serum levels in response to LPS suggests a dysfunction of the macrophages. This dysfunction may be correlated to a reduced initial number of macrophages, to a block in the LPS-induced signaling pathway, or to an increased apoptotic susceptibility of the cIAP2^{-/-} macrophages. However, cIAP2^{-/-} mice have comparable initial cell count numbers of peritoneal and splenic-derived macrophages relative to control littermates as assessed by trypan blue exclusion and Diff Quik™ Stain Kit and flow cytometry (FIG. 4A). The cIAP2^{-/-} mice and wild-type macrophages also stained similarly for the LPS-binding receptor, CD14 (FIG. 4B). Moreover, isolated splenic B cells derived from cIAP2^{-/-} mice proliferated normally in response to varying concentrations of LPS (0.1 to 100 μg/ml) (FIG. 4C). In addition, primary cultures of cIAP2^{-/-} peritoneal macrophages generated comparable levels of TNF-α and IL-1β as littermate controls when exposed to varying doses of LPS (0.1 to 1000 ng/ml) (at 10 and 24 h) (FIGS. 4D, E, F and G in vitro).

cIAP2^{-/-} Macrophages and T Cells are Unresponsive to Anti-Apoptotic LPS and IL-7 Signals

Despite the demonstrated anti-apoptotic properties of cIAP2, purified B cells, T cells, and mouse embryonic fibroblasts from cIAP2^{-/-} mice displayed no significant differences in susceptibility to a variety of apoptotic triggers (α-Fas antibody, C2-ceramide and dexamethasone) in vitro, compared to wild-type cells (data not shown). However, since cIAP2 is strongly inducible by NF-κB activation, it could be argued that the anti-apoptotic properties of cIAP2 could be observed only under appropriate conditions. Macrophages exposed to LPS normally show an increased vigor and resistance towards various apoptotic triggers (3). Indeed, macrophages derived from wold-type mice and pre-treated with LPS showed the expected resistance to Fas-induced death when compared to wild-type derived macrophages that were not pre-exposed to LPS (FIG. 5A) as observed by TUNEL staining. In sharp contrast, macrophages derived from cIAP2^{-/-} mice displayed no difference in their ability to resist Fas-mediated killing with or without pre-exposure to LPS (FIG. 5A). This suggests that LPS-induced up-regulation of cIAP2, within a macrophage, was the protective component against Fas mediated death and is in agreement with Cui et al (3) who demonstrated in vitro that cIAP2 upregulation was at least in part responsible for the general anti-apoptotic resistance displayed by an LPS-induced mcrophage. A similar dependency on cIAP2 was observed with another key regulatory immune cell, the T cell (20). T cells derived from wild-type littermates and pre-exposed to IL-7 (5 or 10 ng/ml) showed a dose dependent resistance against dexamethasone-induced death (FIG. 5B). In contrast, T cells derived from cIAP2^{-/-} mice with or without IL-7 pretreatment displayed no difference in the resistance to apoptosis (FIG. 5B).

cIAP2^{-/-} Macrophages Display an Increased Sensitivity to Apoptosis

We have demonstrated that macrophages derived from cIAP2^{-/-} mice: (1) had typical initial cell count numbers compared to control littermates; (2) produced normal levels of pro-inflammatory cytokine in response to LPS, in vitro; and (3) were highly susceptible to apoptotic triggers relative to control littermates when activated by LPS. It was also found that both cIAP2^{-/-} and control littermates were equally sensitive to PAF, an inflammatory mediator that acts downstream of the LPS-induced activation of macrophages. Given these observations, we predicted that cIAP2^{-/-} mice were resistant to endotoxic shock due to the inability of the cIAP2^{-/-}-derived macrophages to upregulate cIAP2^{-/-}; thus, leading to a loss of visibility and hence a loss of the ability of the cIAP2^{-/-} to produce a lethal inflammatory response. Therefore, in cIAP2^{-/-} mice injected with a normal lethal dose of LPS (35 mg/kg) the expected results would be either a rapid loss of the macrophage populations and/or an increased apoptotic state of macrophages from cIAP2^{-/-} mice relative to control littermates.

To determine the apoptotic sensitivity of cIAP2^{-/-} macrophages within an LPS-induced pro-inflammatory environment, the peritoneal macrophage cell numbers before and during an endotoxin-elicited response were assessed by trypan blue exclusion, Diff Quik™ staining kit and flow cytometry (using the macrophage marker PE conjugated α-F4/80 antibody). In addition, the apoptotic status of the peritoneal and splenic macrophages derived from LPS-injected cIAP2^{-/-} and littermate control mice were also assessed via flow cytometry (using the macrophage marker PE conjugated α-F4/80 antibody and FITC conjugated Annexin V). cIAP2^{-/-} mice demonstrated a markedly reduced number of peritoneal-derived macrophages at 5 h post-LPS injection relative to littermate controls (FIG. 5C). However, total lymphocyte cell count numbers (B and T cells) were comparatively unaffected in both animal types (FIG. 5D). In addition, at 5 hours post LPS injection both the peritoneal and splenic macrophages from cIAP2^{-/-} mice stained ˜100% positive for Annexin V (Table 5). Therefore, peritoneal and splenic macrophages from cIAP2^{-/-} mice undergoing LPS-induced endotoxic shock were highly sensitive to apoptotic stimuli compared to control littermates, in vivo. Liver-derived macrophages are key mediators of LPS-induced endotoxic shock (7, 12). Therefore, to further investigate the survival status of macrophages within LPS treated cIAP2^{-/-} mice we examined cell count levels of liver-derived macrophages within liver sections. Macrophage cell count numbers within both cIAP2^{-/-} and control mice were similar at 2 hours post-IP LPS treatment (FIG. 6A), 235+/−35 and 271+/−29, respectively (n=5, average of 3 sections (average of 5 fields per section) per mouse, P<0.01). However, at the 6 hour time point (FIG. 6B), there was a precipitous drop of liver-derived macrophages of the cIAP2^{-/-} mice, 21+/−12 versus that of the littermates, 148+/−19 (n=5, average of 3 sections (average of 5 fields per section) per mouse, P<0.01). Therefore, like peritoneal and splenic macrophages, the liver-derived macrophages from cIAP2^{-/-} are highly sensitive to LPS in vivo.

II Therapeutic Applications

Despite extensive efforts to formulate an effective treatment for sepsis, mortality rates remain exceptionally high. Many therapies to date have proven to be neurotoxic or have been shown to block only one of the two main inflammatory cytokines, IL-1β or TNF-α. Our results suggest that antagonizing cIAP2 expression and/or function may have a therapeutic benefit for patients with sepsis. Neither IL-1β- nor TNF-α-deficient mice alone are resistant to LPS-induced endotoxic shock (4, 15, 18). The ablation of cIAP expression results not only in a loss of sustained IL-1β production, but also that of TNF-α. Therefore, pharmacological ablation of cIAP2 will limit the severity of inflammatory diseases by transiently abolishing IL-1β- and TNF-α-producing macrophages. The discoveries described herein may be extended to other macrophage-dependent inflammatory disorders, such as, for example, colitis. Our results suggest that cIAP2 is a highly regulated protein whereby its apoptotic inhibitory properties can be observed only under a suitable situation. In addition, the cIAP2-inducing agents, LPS and IL-7, impart their target cells, macrophages and T cells, respectively, with an increased apoptotic resistance. More importantly, cIAP2-null macrophages and T cells are unable to respond to these protective signals indicating that cIAP2 is the crucial protective component.

One such situation occurs when a systemic LPS-activation of the host macrophage population specifically upregulates cIAP2 via NF-kB activation. This response is required in order to resist the intrinsic apoptotic stress by pro-inflammatory cytokines, thereby allowing for the survival of the resident macrophage population and conserving functionality. Consequently, resident macrophages are able to initiate a septic cascade. A lack of cIAP2 sensitizes macrophages to apoptotic stresses, thereby eliminating most of the resident population. This would leave the host animal incapable of inducing septic shock and more importantly, the host animal may not be able to efficiently eliminate a localized infection. Therefore broadly speaking, the present invention provides a method for treating an inflammatory disorder, particularly sepsis, in humans. Treatment of the human subject with an antagonist of cIAP2 expression and/or function, causes otherwise apoptosis resistant cells, such as macrophages, to undergo apoptosis and to reduce or essentially eliminate the inflammatory cascade associated with sepsis. Antagonists of cIAP2 expression and/or function, or regulating cIAP2 promoter or NFkB antagonists may be useful in a monotherapy for the prevention and treatment of IL-1-, apoptosis-, and IFN-mediated diseases, including inflammatory diseases, autoimmune diseases, destructive bone disorders, proliferative disorders (for example, cancer, including solid tumors (lung, CNS, colon, kidney, and pancreas, and the like), infectious diseases, degenerative diseases, necrotic diseases, and the like. Examples of inflammatory disorders include, but are not limited to, inflammatory peritonitis, osteoarthritis, acute pancreatitis, chronic pancreatitis, asthma, adult respiratory distress syndrome, glomerulonephritis, rheumatoid arthritis, systemic lupus erythematosus, scleroderma, chronic thyroiditis, Graves' disease, autoimmune gastritis, insulin-dependent diabetes mellitus (Type I), autoimmune hemolytic anemia, autoimmune neutropenia, thrombocytopenia, chronic active hepatitis, myasthenia gravis, inflammatory bowel disease, Crohn's disease, psoriasis, atopic dermatitis, graft vs. host disease, osteoporosis, multiple myeloma-related bone disorder, leukemias and related disorders, myelodysplastic syndrome, acute myelogenous leukemia, chronic myelogenous leukemia, metastatic melanoma, Kaposi's sarcoma, multiple myeloma, sepsis, septic shock, Shigellosis, Alzheimer's disease, Parkinson's disease, cerebral ischemia, myocardial ischemia, spinal muscular atrophy, multiple sclerosis, AIDS-related encephalitis, HIV-related encephalitis, aging, alopecia, neurological damage due to stroke, ulcerative collitis, infectious hepatitis, juvenile diabetes, lichenplanus, acute dermatomyositis, eczema, primary cirrhosis, uveitis, Behcet's disease, atopic skin disease, pure red cell aplasia, aplastic anemia, amyotrophic lateral sclerosis, nephrotic syndrome, burns, bronchitis, tendinitis, bursitis, periarteritis nodosa, thyroiditis, Hodgkin's disease, rheumatic fever, sarcoidosis, polymyositis, gingivitis, hypersensitivity, conjunctivitis, swelling occurring after injury, allergic rhinitis, endotoxin shock syndrome, and atherosclerosis, psoriatic arthritis, vasculitis, Polymyalgia, Rheumatica, Wegener's granulomatosis, temporal arteritis, chronic obstructive pulmonary disease, cryoglobulinemia, transplant rejection and ataxia telangiectasia. Also contemplated in the methods of the present invention are systemic diseases or diseases with effects localized in the liver or other organs having an inflammatory or apoptotic component caused by excess dietary alcohol intake or viral infections, such as HIV, influenza, Epstein-Barr, cytomegalovirus, herpes simplex virus, HBV, HCV, HGV, yellow fever virus, dengue fever virus, and Japanese encephalitis virus.

Also contemplated is the use of transplantation a method for treating sepsis in a subject. Transplantation may involve administering (transducing) directly to the cell a disrupted ciap2 gene so as to disrupt cIAP2 protein expression and/or function in the cell, and then transplanting the cells into the recipient subject. The transplantation may involve administering a cell into the recipient by injecting a cell suspension into the recipient. Also, another method of transplantation may involve culturing the cell to be transplanted before administration to the recipient.

III Screening Assay

Transgenic non-human mammals of the present invention may be used to screen for compounds useful in the treatment of sepsis or septic shock. Contemplated is such a method that involves applying a sepsis or septic shock challenge to a transgenic non-human mammal with a disrupted ciap2 gene and exhibiting resistance to septic shock and then administering a test compound to the transgenic or wild type mammal. The test compound may be for example derived from a compound library and the like. The effect of the test compound on the susceptibility to manifestation of sepsis or septic shock in the mammal may then be determined followed by correlating the effect of the test compound on septic shock or sepsis of the mammal with an effect of the test compound on the macrophages in a non treated mammal having a disrupted ciap2 gene, or in a wild type mammal with a normal phenotype.

IV Kits

The present invention also contemplates an article of manufacture in the form of a kit for use in testing the sensitivity of macrophages to apoptosis stimuli. The kit would typically include, packaged together, a vessel or vessels, such as a vial, for receiving a sample of macrophages from the transgenic mammal, after a septic shock or sepsis challenge; a sterile needle for drawing the blood from the mammal; a stain for staining the macrophages for Annexin V; and instructions for comparing the stained macrophages with a control mammal. The kits can be manufactured according to the specific inflammatory disorder for which the sample is to be taken. The instructions can describe the steps necessary to take appropriate blood from the mammal, and how to mix the apoptosis stimuli, the stain and the blood sample.

### EXAMPLES

The present invention is further illustrated by the following non-limiting examples:

1. Generation of Germline Chimeras and Homozygous Mice.

129/sv genomic clones (13) spanning the mouse ciap2 gene were used to construct a replacement type targeting vector in which an IRES-lacZ and phosphoglycerate kinase (PGK)-neomycin (neo) cassette (SA-IRES-βgeo; (16)) replaced exons 2 to 5 in the plasmid pKO (Holcik and Korneluk, unpublished). The resulting targeting vector (pKO.hiap1) was comprised of a 4.1 kb 5′ arm and a 5.5 kb 3′ arm bracketing the IRES-lacZ/PGK-neo insertion. RW4 embryonic tem (ES) cells were electroporated as described (28) and DNA from neomycin resistant clones was extracted and analyzed. Disruption of the ciap2 allele was confirmed by Southern blot analysis of EcoRV digested genomic DNA after hybridization with a probe corresponding to exon 1 of the ciap2 gene. Chimeric mice were produced by morula aggregation (27) with targeted RW-4 cells. Chimeric male progeny were mated with 129/SvJ females and heterozygous progenies were backcrossed to C57BL/6 mice for at least 10 generations. Heterozygous mice were then crossed to produce homozygous cIAP^{-/-} mice. Both the electroporation of ES cells and the generation of chimeric animals were performed at the Genome Systems Inc. facility (St. Louis, Mo.). Mice were housed in a specific pathogen-free environment and all experiments were performed in accordance with the guidelines of the Canadian Council on Animal Care.

2. Southern Blot Analysis.

Genomic DNA was isolated by standard methods and digested with EcoRV, separated on agarose gels, and transferred to Biodyne Nylon Paper (Life Technologies, Rockville, Md.). Full length ³²P-labeled cIAP2 cDNA probes were prepared by using Rediprime (Amersham Pharmacia) and ³²P-dCTP (Amersham Pharmacia) according to the manufacturer's directions. Membranes were washed with 0.1×SSC/0.1% SDS at 65° for 10 minutes and exposed to X-ray film.

3. Western Blot Analysis.

Mouse tissue was weighed and subsequently lysed in five volumes(w/v) of lysis buffer (50 mM Tris.HCl, pH 7.4, 150 mM NaCl, 1 mM EDTA, 1 mM vanadate, 1% (v/v) Nonidet P-40, 0.25% (v/v) sodium deoxycholate, 1 μg/ml leupeptin, 1 μg/ml aprotinin, 1 μM phenylmethylsulphonyl fluoride) and then crushed and well mixed. The samples were then rotated for 45 minutes at 4° C. The samples were then centrifuged for 15 minutes at ˜14,000 rpm, in a microcentrifuge. The supernatant was collected and assayed by BCA kit (Pierce) and equal amounts of protein sample of S/N lysate were loaded per lane and separated on a SDS/PAGE gel and analyzed by Western blotting by using in house prepared rabbit polyclonal α-cIAP2, αCIAP1 or α-XIAP antibodies (1:2500 dilution), followed by anti-rabbit HRP-conjugated secondary antibody (Amersham) and the immune complexes were visualized using an enhanced chemiluminescence kit (Roche).

4. mRNA Isolation and Quantitative mRNA Analysis.

mRNA was extracted from mouse embryonic fibroblasts or peritoneal macrophages using Qiagen Rneasy 96 wells extraction kit (Qiagen, Mississauga, ON, Canada) and run on a TaqMan® instrument (Perkin-Elmer, Foster City, Calif.) using specific DNA probes for murine xiap, ciap1 and 2 and the TaqMan® EZ RT-PCR kit (Qiagen, Mississauga, ON, Canada).

5. Animal LPS Models.

Adult 4-6 week old mice (n=6 to 10) were injected intraperitoneally (i.p.) with a range of LPS doses (10 to 200 mg LPSD/kg) from E. coli (Sigma) in a total volume of 0.2 ml nonpyrogenic saline.

Adult 4-6 week old mice (n=3), cIAP2^{-/-} and littermate control mice, were injected i.p. with a LD₁₀₀ dose of LPS (35 mg LPS/kg). At the appropriate times mice were anesthetized with phenobarbital and killed by cervical dislocation. The plasma was then collected from these animals and used to determine serum concentrations of IL-1β, TNF-α and IL-12 via an ELISA kit (R & D Systems).

Adult 4-6 week old mice (n=6) were injected i.p. with LPS (35 mg LPS/kg) at time 0 and 5 hours the mice were euthanized and the cells of the peritoneum were collected and stained to determine macrophage, T and B cell percentages. The macrophages were also stained with FITC labeled Annexin V (Immunotech, Marseille, France) to determine apoptotic status.

7. Flow Cytometry.

T and B cells were isolated from mouse lymphoid tissue (spleen) by first mincing and then pressing the tissue through a 10 μm metal mesh and were then counted by using trypan blue exclusion method. Macrophages were harvested from mice using an inter-peritoneal (i.p.) lavage (3×10 of media: 5% FCS, 50 μM β-mercaptoethanol, 125 mM L-glutamine, penicillin and streptomycin, at 4° C.) and washed once (centrifuged at 800 Xg for 15 min) and resuspended at ˜2×10⁷ cells/ml for peritoneal macrophages. Subsequently, peritoneal macrophages were layered on 5 ml of room temperature Lympholyte-M (CedarLane, Canada) and centrifuged at 1500×g for 20 min at room temperature. For splenic macrophages 3 ml of the minced and pressed tissue (˜2×10⁷ splenocytes/ml) was layered on 5 ml of room temperature Lympholyte-M (CedarLane, Canada) and centrifuged at 1500×g for 20 min at room temperature. Subsequently, either peritoneal or spleen-derived macrophages were collected from the interface layer, washed twice with complete DMEM and re-suspend in 1 ml complete DMEM. The resulting cells were then counted by using typan blue exclusion and Diff Quik™ Stain Kit (IMEB INC, San Marcos, Calif.). Cells (10⁵-10⁶) were incubated with the following conjugated monoclonal antibodies: α-CD3ε-flourescein isohiocyanate (FITC), CD4-phycoerythin (PE), CD8α-Cy-Chrome, CD69-PE; B229-FITC, B220-PE, CD11b-Cy-Chrome, (Pharmigen) F4/80-FITC and F4/80PE (Cedarlane Laoratories, Hornby, ON, Canada). Flow cytometric analyses were performed on a Coulter XL cytometer (Coulter, Canada). Concentrations of TNF-α and IL-1β, in primary tissue culture supernatants were determined by ELISA kit (R&D Systems).

8. Primary Tissue Culture and Death assays.

Primary cultures were maintained in DMEM supplemented with: (for macrophages) 10% FCS, 10 ng/ml granulocyte-macrophage colony stimulating factor (GM-CSF) (R&D Systems), (for T cells, B cells and thymocytes) 5% FCS, 50 μM β-mercaptoethanol, 125 mM L-glutamine, penicillin, and streptomycin (˜85% confirmed via a FITC-stained anti-CD3 antibody using flow cytometry).

Confluent primary cultures of peritoneal-derived macrophages were either pre-treated with LPS (10 μg/ml, 4 h) or not pre-treated prior to exposure to α-Fas antibody (20 μg/ml, clone Jo2) and then TUNEL stained (Roche) to assess cell viability. Primary cultures of spleen derived T cells were pre-incubated with a range of IL-7 concentrations (0, 5, and 10 ng/ml) and then exposed to dexamethasone (100 nM) and T cell survival was monitored over a 12 h period.

TABLE 1
Survival of cIAP2^{-/-} and control mice
after treatment with α-Fas antibody

| | | Average survival time |
|---|---|---|
| Mice | Survival | (hours) |
| CIAP2^{-/-} mice | 0/6 | 3.3 ± 0.9 |
| CIAP2^{-/+} mice | 0/6 | 3.7 ± 0.9 |
| CIAP2^{+/+} mice | 0/6 | 3.1 ± 0.8 |

Mice (406 weeks of age) were injected intraperitoneally with α-fas antibody (100 μg, 0.2 ml, cloneJo2)

TABLE 2
Survival of cIAP^{-/-} and control mice after
treatment with platelet-activating factor (PAF).

| | PAF Concentration | | | | | |
|---|---|---|---|---|---|---|
| | 0 | 1 | 2 | 2.5 | 3 | 5 |
| Mice | μg/kg | μg/kg | μg/kg | μg/kg | μg/kg | μg/kg |
| cIAP^{-/-} mice | 6/6 | 6/6 | 6/6 | 6/6 | 0/6 | 0/6 |
| cIAP2+/+ mice | 6/6 | 6/6 | 6/6 | 6/6 | 0/6 | 0/6 |

Mice (4-6 weeks of age) were injected intravenously with the indicated doses of PAF in 0.2 ml nonpyrogenic saline. All deaths occurred within 24 h.

TABLE 3
Survival of cIAP2−/− and control mice
treatment with D-galactosamine and LPS.

| | LPS Concentration | | | | | |
|---|---|---|---|---|---|---|
| | 0 | 0.5 | 1 | 5 | 10 | 20 |
| Mice | μg/kg | μg/kg | μg/kg | μg/kg | μg/kg | μg/kg |
| cIAP2^{-/-} mice | 6/6 | 6/6 | 6/6 | 2/6 | 0/6 | 0/6 |
| cIAP2^{+/+} mice | 6/6 | 6/6 | 6/6 | 2/6 | 0/6 | 0/6 |

Mice (4-6 weeks of age) were injected intraperitoneally with the indicated does of LPS (E. coli K235) with D-galactosamine (0.6 g/kg) in 0.2 ml nonpyrogenic saline. All deaths occurred within 6 hours.

TABLE 4
cIAP2^{-/-} mice cytokine serum levels are attenuated compared to cIAP2^{+/+}
mice after bot6h received an i.p. LPS does of 35 mg/kg.

| | Time | | | | | |
|---|---|---|---|---|---|---|
| | 2 hours | | 6 hours | | 10 hours | |
| | Mice | | | | | |
| | cIAP2^{-/-} | cIAP2^{+/+} | cIAP2^{-/-} | cIAP2^{+/+} | cIAP2^{-/-} | cIAP2^{+/+} |
| | mice | mice | mice | mice | mice | mice |
| IL-1β | 314 ± 153 | 476 ± 24 | 476 ± 78 | 993 ± 158 | 162 ± 21 | 669 ± 48 |
| Serum | | | | | | |
| Levels | | | | | | |
| (pg/ml) | | | | | | |
| TNFα | 1589 ± 413 | 1623 ± 348 | 202 ± 89 | 502 ± 56 | 8 ± 7 | 371 ± 64 |
| Serum | | | | | | |
| Levels | | | | | | |
| (pg/ml) | | | | | | |
| IL-12 | 152 ± 24 | 210 ± 17 | 11 ± 5 | 260 ± 31 | | |
| Serum | | | | | | |
| Leves | | | | | | |
| (pg/ml) | | | | | | |

n = 6; results are mean ± s.d.

TABLE 5
cIAP2^{-/-}-derived macrophages are highly sensitive
to apoptotic stimuli during endotoxic shock, in vivo

| | cIAP2^{-/-} mice | | cIAP2^{+/+} mice | |
|---|---|---|---|---|
| | Percent | Percent | Percent | Percent |
| Mice | Macrophage | apoptotic | macrophage | Apoptotic |
| Peritoneal cavity | 41 ± 24 | 15 ± 3 | 54 ± 29 | 10 ± 2 |
| (At Time: 0 hrs | | | | |
| post-LPS) | | | | |
| Peritoneal cavity | 8 ± 11 | 100 ± 3 | 31 ± 17 | 66 ± 8 |
| (At Time: 5 hrs | | | | |
| post-LPS) | | | | |
| Spleen | 24 ± 11 | 2 ± 5 | 33 ± 15 | 5 ± 3 |
| (At Time: 0 hrs | | | | |
| post-LPS) | | | | |
| Spleen | 24 ± 8 | 98 ± 4 | 28 ± 5 | 46 ± 8 |
| (At Time: 0 hrs | | | | |
| post-LPS) | | | | |

The percentage of apoptotic peritoneal- and splenic-derived macrophages from either cIAP2^{-/-} or cIAP2^{+/+} mice (n = 6; results are mean ± s.d.) that were injected i.p. with LPS.

Discussion

Our data demonstrates that upon a bolus IP injection of LPSD, macrophage survival is dependent upon rapid upregulation of the cIAP protein. LPS activation of peritoneal-derived macrophages induced a rapid and dramatic increase of ciap2 message 2o times above untreated controls in less than one hour. Moreover, in contrast to published reports of the XIAP ^{-/-} mice (5) there was no observed general compensatory increases in either mRNA or protein levels of the other IAP family members, cIAP1 and XIAP.

LPS Confers Apoptotic Resistance to Macrophages via Induction of cIAP2 Protein

The observed rapid induction cIAP2 in macrophages in response to LPS activation suggested that, at least in part, cIAP2 might be a key resistance component for maintaining macrophage viability under apqptotic conditions. Indeed, the in vivo work presented here demonstrates that cIAP2 has a critical anti-apoptotic role in sustaining macrophage viability. Peritoneal macrophages derived either from cIAP2^{-/-} or control littermates displayed similar sensitivity to Fas-induced death, however, when pretreated with LPS only macrophages from cIAP2^{+/+} mice, displayed an increased resistance to Fas-induced apoptosis.

Ablation of cIAP2 Renders Macrophages Susceptible to Apoptosis During Endotoxic Shock In Vivo

We have shown that LPS activation of macrophages induces a potent and robust induction of cIAP2. Moreover, loss of cIAP2 protects mice from acute endotoxic shock, and this is associated with the effects of cIAP2 loss on the deaths of macrophages that normally produce large amounts of pro-inflammatory cytokines. In support of this notion, peritoneal macrophage numbers from cIAP2^{-/-} mice reduced considerably by the 5 hour time point post LPS injection, relative to cIAP2^{-/-} mice at time 0 hours. More importantly, peritoneal macrophage numbers from cIAP2^{-/-} mice are lower by the 5 hour time point post LPS injection, relative to wild-type mice at the same time point. In addition, the peritoneal and splenic macrophages from cIAP2^{-/-} mice 5 hours post LPS injection were ˜100% apoptotic for both macrophage populations.

cIAP2^{-/-}-Derived Macrophages are Normal in Response to LPS Treatment, In Vitro

The loss of cytokine production in cIAP2^{-/-} mice treated with a lethal dose of LPS may be due to a signaling dysfunction of the macrophages. Similar to the results obtained for cIAP2^{-/-} mice, TLR-4-deleted (8) and MyD88-deficient mice (9) have also been found to be resistant to LPS-induced endotoxic shock. However, B cells isolated from these animals failed to proliferate in response to LPS, whereas B cells from cIAP2^{-/-} mice responded normally to LPS. Furthermore, cultured macrophages derived from either TLR4- or MyD88-null mice were unable to produce pro-inflammatory cytokines. In the case of TLR4 and MyD88 deficiency, the observed resistance to endotoxic shock is due to a block of the LPS-induced activation pathway of the macrophage. In contrast, cIAP2^{-/-} mice macrophages exposed to LPS generate normal levels of TNF-α and IL-1β suggesting that the classical LPS-induced NF-κB pathway is intact in macrophages lacking cIAP2.

Proposed Mechanism of Action: cIAP2^{-/-} Mice Resist Endotoxic Shock

Without wishing to be bound by theory, we believe that LPS challenge of mice causes the activation of multiple types of genes, including the upregulation of the survival genes. The pro-inflammatory response generates an inherently hostile environment that can be lethal to both pathogen and host immune cell. Therefore, expression of pro-survival genes is likely vital to maintain macrophage viability during an immune response. The inability to upregulate cIAP2 renders LPS-activated macrophages highly susceptible to apoptotic triggers, thereby quickly eliminating the resident macrophage population soon after the initiation of a systemic inflammatory response. This leads to the loss of the principal source of pro-inflammatory cytokines and subsequently to the attenuation of the immune response, preventing the development of multiple organ failure.

Unlike cIAP1 and XIAP, the results presented here indicate that cIAP2 regulation is dependent upon signal transduction pathways. An up-regulation of cIAP2 mRNA was elicited in T cells upon exposure to IL-7 and a dramatic increase was observed in macrophages treated with LPS. There is a possibility that the cIAPs and XIAP may be able to functionally “stand in” for one another. Although the caspases inhibited by the cIAPs coincide, cIAP1 and cIAP2 bind caspases with significantly lower affinities compared to XIAP. In addition, each IAP has unique properties and cellular localizations. XIAP is involved in the TAK1/JNK1 signaling cascade (21) while the cIAPs associate with TRAFs (19). In addition, and more importantly, the observed differences in IAP regulation, demonstrated here, serve to underscore the non-redundant physiological functions of the IAPs and indicate that these proteins cannot functionally substitute each other.

9. Administering of cIAP2 Antagonists to Human Patients Suffering from Sepsis.

The diagnosis of severe sepsis in human requires the presence of a presumed or known site of infection, evidence of a systemic inflammatory response, and an acute sepsis-associated organ dysfunction. Specific diagnostic criteria used in past clinical trials to define patients with severe sepsis include the following:

1) A presumed or known site of infection is indicated by one of the following:
a) Purulent sputum or respiratory sample, or a chest radiograph with new infiltrates not explained by a noninfectious process
b) Spillage of bowel contents noted during an operation
c) Radiographic or physical examination evidence of an infected collection
d) White blood cells in a normally sterile body fluid
e) Positive blood culture
f) Evidence of infected mechanical hardware by physical or radiographic examination
   2) Evidence of a systemic inflammatory response is indicated by at least two of the following:
a) Fever or hypothermia—Core body temperature of greater than or equal to 38° C. or less than or equal to 36° C.
b) Tachypnea—greater than or equal to 20 breaths per minute or need for mechanical ventilation for an acute process
c) Tachycardia—heart rate greater than or equal to 90 beats per minute, unless the patient has a preexisting tachycardia
d) White blood cell count—greater than or equal to 12,000 cells/mm³ or less than or equal to 4,000 cells/mm³, or greater than 10% bands on differential
   3) A sepsis-induced organ failure is indicated by one of the following criteria:
a) Cardiovascular dysfunction—mean arterial pressure less than or equal to 60 mm Hg, the need for vasopressors to maintain this blood pressure in the face of adequate intravascular volume (central venous pressure greater than 8 or pulmonary artery occlusion pressure greater than 12), or after an adequate fluid challenge has been given
b) Respiratory organ failure—an arterial oxygen pressure/fraction of inspired oxygen ratio less than 250 in the absence of pneumonia or less than 200 in the presence of pneumonia
c) Renal dysfunction—urine output less than 0.5 mL/kg/hr for 1 hour in the face of adequate intravascular volume or after an adequate fluid challenge
d) Hematologic dysfunction—thrombocytopenia with 80,000 platelets/mm³, a 50% drop in the previous 3 days, or a prothrombin-INR greater than 1.2 that cannot be explained by liver disease or concomitant warfarin usage
e) Unexplained metabolic acidosis—a pH less than 7.30 and a plasma lactate greater than 1.5 times the upper limit of normal for the laboratory

A double blind placebo controlled clinical trial in human patients suffering from sepsis is used in which the patients are treated with the placebo or a cIAP2 antagonist. The antagonist is administered by continuous intravenous infusion and reduction or elimination of the severity of any of the above symptoms indicates the treatment of sepsis.

### REFERENCES

1) Beutler, B. (2000). TIr4: central component of the sole mammalian LPS sensor. Curr Opin Immunol 12, 20-26.
2) Cannon, J. G., Tompkins, R. G., Gelfand, J. A., Michie, H. R., Stanford, G. G., van der Meer, J. W., Endres, S., Lonnemann, G., Corsefti, J., Chernow., B., and et al (1990) Circulating interleukin-1 and tumor necrosis factor in septic shock and experimental endotoxin fever, J Infect Dis 161, 79-84.
3) Cui, X., Imaizumi, T., Yoshida, H., Tanji, K., Matsumiya, T., and Satoh, K., (2000). Lipopolysaccharide induces the expression of cellular inhibitor of apoptosis protein-2 in human macrophages, Biochim Biophys Acta 1524, 178-182.
4) Fantuzzi, G., Zheng, H., Faggioni, R., Bnini, F., Ghezzi, P., Sipe, J. D., Shaw, A. R., and Dinarello, C. A. (1996). Effect of endotoxin in IL-1 beta-deficient mice, J Immunol 157, 291-296.
5) Harlin, H., Reffey, S., Duckett, C. S., Lindsten, T., and Thompson, C. B. (2001) Characterization of XIAP-deficient mice, Mol Cell Biol 21, 3604-3608. Hashimoto, S., Morohoshi, K., Suzuki, T., and Matsushima, K., (2003) Lipopolysaccharide-inducible gene expression profile in human monocytes. Scand J Infect Dis 35,619-627.
6) Haziot, A., Chen, S., Ferrero, E., Low, M. G., Silber, R., and Goyert, S. M. (1988). The monocyte differentiation antigen, CD14 is anchored o the cell membrane by a phosphatidylinositol linkage. J Immunol 141, 547-552.
7) Hirano, K., Shimizu, Y., Nakyama, Y., Minemura, M., Yasumura, S., Sugiyama, T. (2005). Overexpression of granulocyte-marcophage colony-stimulating factor in mouse liver enhances the susceptibility of lipopolysaccharide leading to massive of hepatocytes. Liver Int 25:1027-35.
8) Hoshino, K., Takeuchi, O., Kawai, T., Sanjo, H., Ogawa, T., Takeda, Y., Takeda, K., and Akira, S. (1999). Cutting edge: Toll-like receptor 4 (TLR4)-deficient mice are hyporesponsive to Lipopolysaccharide: evidence for TLR4 as the Lps gene product. J Immunol 162, 3749-3752.
9) Kawai, T., Adachi, O., Ogawa, T., Takeda, K., and Akira, S. (1999). Unresponsiveness of MyD88-deficient mice to endotoxin. Immunity 11, 115-122.
10) Koay, M. A., Gao, X., Washington, M. K., Parman, K. S., Sadikot, R. T., Blackwell, T. S., and Christman, J. W. (2002). Macrophages are necessary for maximal nuclear factor-kappa B activation in response to endotoxin. Am J Respir Cell Mol Biol 26, 572-578.
11) Kumar, A., Thota, V., Dee, L., Olson, J., Uretz, E., and Parillo, J. E. (1996). Tumor necrosis factor alpha and interleukin 1 beta are responsible for in vit ro myocardial cell depression induced by human septic shock serum. J Exp Med 183, 949-958.
12) Kumins, N. H., J. Hunt, R. L. Gamelli, and J. P. Filkins. 1996. Partial hepatectomy reduces the endotoxin-induced peak circulating level of tumor necrosis factor in rats. Shock 5:385-8.
13). Liston, P, Lefebvre, C., Fong, W. G., Xuan, J. Y., and Korneluk, R. G., (1997). Genomic characterization of the mouse inhibitor of apoptois protein1 and 2 genes, Genomics 46:495-503.
14) Liston, P., Roy, N., Tamai, K., Lefebvre, C., Baird, S., Cherton-Horvat, G., Farahani, R., McLean, M., Ikeda, J. E., MacKenzie, A., and Korneluk, R. G. (1996). Suppression of apoptosis in mammalian cells by NAIP and a related family of IAP genes. Nature 379, 349-353.
15) Marino, M. W., Dunn, A., Grail, D., Inglese, M., Noguchi, Y., Richards, E., Jungbluth, A., Wada, H., Moore, M., Williamson, B., et al (1997). Characterization of tumor necrosis factor-deficient mice. Proc Natl Acad Sci USA 94, 8093-8098.
16) Mountford, P., Zevnik, B., Duwekm A., Nichols, J., Li, M., Dani, C., Robertson, M., Chambers, I., and Smith, A. (1994). Dicistonic targeting constructs: reporters and modifiers of mammalian fene expression. Proc Natl Acad Sci USA 91:4303-7.
17) Park, S. M., Yoon, J. B., and Lee, T. H., (2004). Receptor interatcting protein is ubiquitinated by cellular inhibitor of apoptosis proteins (c-IAP1 and cIAP2) in vitr5o. FEBS Letters 566:151-156.
18) Riedemann, N. C., Guo, R. F., and Ward, P. A. (2003) The enigma of sepsis. J Clin Invest 112:460-7.
19) Rothe, M., Pan, M. G., Henzel, W. J., Ayres, T. M., and Goeddel, D. V. (1995). The TNFR2-TRAF signaling complex contains two novel proteins related to baculoviral inhibitor of apoptosis proteins. Cell 83, 1243-1252.
20) Sade, H., and Sarin, A., (2003). IL-7 inhibits dexamethasone-induced apoptosis via Akt/PKB in mature, peripheral T cells, Eur J Immunol 33, 913-919.
21) Sanna, M. G., Duckett, C. S., Richter, B. W., Thompson, C. B. and Ulevitch, R. J. (1998). Selective activation of JNK1 is necessary for the anti-spoptotic activity of hILP. Proc Natl Acad Sci USA 95:6015-20.
22) Schoemaker, M. H., Ros, J. E., Homan, M., Trautwien, C., Poelstra, K., van Goor, H., Jansen, P. L., and Moshage, H. (2002). Cytokine regulation of pro and anti-apoptotic genes in rat hepatocytes NF-kappaβ-regulated inhibitor of apoptosis protein 2 (cIAP2) prevents apoptosis. J Hepatol 36, 742-750.
23) Sriskandan, S., and Cohen, J., (1995). The pathogenisi of septic shock. J Infec 30, 201-206.
24) Sun, X. M., and Hsuch, W. (1991). Platelet-activating factor produces shock, in vivo complement activation, and tissue injury in mice. J Immunol 147, 509-514.
25) Wang, C. Y., Mayo, M. W., Korneluk, R. G., Goeddel, D. V., and Baldwin, A. S., Jr. (1998). NF-kappaβ antiapoptosis: induction of TRAF1 and TRAF2 and c-IAP1 and C-IAP2 to suppress caspase-8 activation. Science 281, 1680-1683.
26) Weighardt, H., Kaiser-Moore, S., Vabulas, R. M., Kirschning, C. J., Wagner, H., and Holzmann, B., (2002). Cutting edge: myeloid differentiation factor 88 deficiency improves resistance against sepsis caused by polymicrobial infection. J Immunol 169, 2823-2827.
27) Wood, S. A., Allen, N. D., Rossant, J., Auerbach, A., Nagy, A. (1993) Non-injection methods for the production of embryonic stem cell-embryo chimaeras. Nature 365:87-9.
28) Wurst, W., J. A. L. (1993). Production of targeted embryonic stem cell clones, p. 33-62. In J. A. L. (ed.). Gene targeting. Oxford University Press, Oxford New York Tokyo.

### OTHER EMBODIMENTS

From the foregoing description, it will be apparent to one of ordinary skill in the art that variations and modifications may be made to the invention described herein to adapt it to various usages and conditions. Such embodiments are also within the scope of the present invention.

All publications mentioned in this specification are hereby incorporated by reference.

While specific embodiments have been described, those skilled in the art will recognize many alterations that could be made within the spirit of the invention, which is defined solely according to the following claims:

What is claimed is:

1. A method of treating an inflammatory disorder in a subject, the method comprising: administering to the subject in need thereof an antagonist of cIAP2 expression and/or function, thereby treating the disorder.

2. The method, according to claim 1, in which the antagonist antagonizes cIAP2 protein function.

3. The method, according to claim 1, in which the inflammatory disorder is characterized by cells which produce cytokines.

4. The method, according to claim 3, in which the cells comprise macrophages, T-cells, or fibroblasts.

5. The method, according to claim 3, in which the cytokines are IL-1β or TNF-α.

6. The method, according to claim 1, in which the inflammatory disorder is selected from inflammatory peritonitis, osteoarthritis, acute pancreatitis, chronic pancreatitis, asthma, adult respiratory distress syndrome, glomerulonephritis, rheumatoid arthritis, systemic lupus erythematosus, scleroderma, chronic thyroiditis, Graves' disease, autoimmune gastritis, insulin-dependent diabetes mellitus (Type I), autoimmune hemolytic anemia, autoimmune neutropenia, thrombocytopenia, chronic active hepatitis, myasthenia gravis, inflammatory bowel disease, Crohn's disease, psoriasis, atopic dermatitis, graft vs. host disease, osteoporosis, multiple myeloma-related bone disorder, leukemias and related disorders, myelodysplastic syndrome, acute myelogenous leukemia, chronic myelogenous leukemia, metastatic melanoma, Kaposi's sarcoma, multiple myeloma, sepsis, septic shock, Shigellosis, Alzheimer's disease, Parkinson's disease, cerebral ischemia, myocardial ischemia, spinal muscular atrophy, multiple sclerosis, AIDS-related encephalitis, HIV-related encephalitis, aging, alopecia, neurological damage due to stroke, ulcerative collitis, infectious hepatitis, juvenile diabetes, lichenplanus, acute dermatomyositis, eczema, primary cirrhosis, uveitis, Behcet's disease, atopic skin disease, pure red cell aplasia, aplastic anemia, amyotrophic lateral sclerosis, nephrotic syndrome, burns, bronchitis, tendinitis, bursitis, periarteritis nodosa, thyroiditis, Hodgkin's disease, rheumatic fever, sarcoidosis, polymyositis, gingivitis, hypersensitivity, conjunctivitis, swelling occurring after injury, allergic rhinitis, endotoxin shock syndrome, and atherosclerosis, psoriatic arthritis, vasculitis, Polymyalgia, Rheumatica, Wegener's granulomatosis, temporal arteritis, chronic obstructive pulmonary disease, cryoglobulinemia, transplant rejection and ataxia telangiectasia.

7. The method, according to claim 6, in which the inflammatory disorder is sepsis.

8. The method, according to claim 1, in which the inflammatory disorder is LPS or IL-7 induced.

9. The method, according to claim 1, in which the subject is human

10. A method of causing apoptosis in cells, the cells being characterized by producing cytokines, the method comprising: antagonizing cIAP2 expression and/or function by contacting the cells with a cIAP2 antagonist, thereby causing the cells to undergo apoptosis.

11. The method, according to claim 10, in which the antagonist antagonizes cIAP2 protein function.

12. The method, according to claim 10, in which the cells are from a subject suffering from an inflammatory disorder, the disorder being characterized by cells which produce cytokines.

13. The method, according to claim 10, in which the cells comprise macrophages, T-cells, or fibroblasts.

14. The method, according to claim 10, in which the cytokines are IL-1β or TNF-α.

15. The method, according to claim 11, the inflammatory disorder is selected from inflammatory peritonitis, osteoarthritis, acute pancreatitis, chronic pancreatitis, asthma, adult respiratory distress syndrome, glomerulonephritis, rheumatoid arthritis, systemic lupus erythematosus, scleroderma, chronic thyroiditis, Graves' disease, autoimmune gastritis, insulin-dependent diabetes mellitus (Type I), autoimmune hemolytic anemia, autoimmune neutropenia, thrombocytopenia, chronic active hepatitis, myasthenia gravis, inflammatory bowel disease, Crohn's disease, psoriasis, atopic dermatitis, graft vs. host disease, osteoporosis, multiple myeloma-related bone disorder, leukemias and related disorders, myelodysplastic syndrome, acute myelogenous leukemia, chronic myelogenous leukemia, metastatic melanoma, Kaposi's sarcoma, multiple myeloma, sepsis, septic shock, Shigellosis, Alzheimer's disease, Parkinson's disease, cerebral ischemia, myocardial ischemia, spinal muscular atrophy, multiple sclerosis, AIDS-related encephalitis, HIV-related encephalitis, aging, alopecia, neurological damage due to stroke, ulcerative collitis, infectious hepatitis, juvenile diabetes, lichenplanus, acute dermatomyositis, eczema, primary cirrhosis, uveitis, Behcet's disease, atopic skin disease, pure red cell aplasia, aplastic anemia, amyotrophic lateral sclerosis, nephrotic syndrome, burns, bronchitis, tendinitis, bursitis, periarteritis nodosa, thyroiditis, Hodgkin's disease, rheumatic fever, sarcoidosis, polymyositis, gingivitis, hypersensitivity, conjunctivitis, swelling occurring after injury, allergic rhinitis, endotoxin shock syndrome, and atherosclerosis, psoriatic arthritis, vasculitis, Polymyalgia, Rheumatica, Wegener's granulomatosis, temporal arteritis, chronic obstructive pulmonary disease, cryoglobulinemia, transplant rejection and ataxia telangiectasia.

16. The method, according to claim 15, in which the inflammatory disorder is sepsis.

17. The method, according to claim 12, in which the inflammatory disorder is LPS or IL-7 induced.

18. The method, according to claim 12, in which the subject is human.

19. A method of treating sepsis in a subject, the method comprising: administering to the subject in need thereof an antagonist of cIAP2 expression and/or function, thereby treating the sepsis.

20. The method, according to claim 19, in which the antagonist antagonizes cIAP2 protein function.

21. The method, according to claim 19, in which the subject is a human.

22. A disrupted ciap2 gene which comprises a nucleic acid sequence, according to SEQ ID NO. 1.

23. A disrupted ciap2 gene which consists of a nucleic acid sequence, according to SEQ ID NO.1.

24. A transgenic non-human mammal comprising the disrupted ciap2 gene, according to claim 22.

25. The transgenic non-human mammal, according to claim 24, is a mouse.

26. A transgenic non-human mammal model for studying sepsis or septic shock, wherein the mammal comprises the disrupted ciap2 gene, according to claim 22.

27. A transgenic non-human mammal model for studying infection, wherein the mammal comprises the disrupted ciap2 gene, according to claim 22.

28. A transgenic non-human mammal whose genome is heterozygous for a disruption in the ciap2 gene, according to claim 22, wherein the disruption in a homozygous state inhibits the production of function cIAP2 protein, which results in a transgenic non-human mammal having a reduced severity of sepsis as compared to a wild type mammal.

29. A transgenic non-human mammal whose genome is homozygous for the disrupted ciap2 gene, according to claim 22, the disrupted gene in a homozygous state inhibiting the production of functional cIAP2 protein, which results in a transgenic non-human mammal having a reduced severity of sepsis as compared to a wild type mammal.

30. A cell which is isolated from the transgenic non-human mammal, in which the genome of the cell comprises the homozygous disrupted ciap2 gene, according to claim 22, wherein the disruption of the ciap2 gene inhibits production of functional cIAP2 protein.

31. A primordial germ cell which is isolated from a transgenic non-human mammal embryo whose genome comprises a homozygous disrupted ciap2 gene, according to claim 22, wherein the disruption of the ciap2 gene inhibits production of functional cIAP2 protein.

32. A cell line comprising a progeny of the cell, according to claim 30, wherein the progeny of the cell comprise a homozygous disrupted ciap2 gene, wherein the disruption inhibits production of functional cIAP2 protein.

33. A method for producing a heterozygous non-human mammal, the mammal having somatic and germ cells containing a gene coding for a disrupted mammal cIAP2 protein, according to claim 22, the method comprising:
(a) introducing the gene into an embryo blastocyst;
(b) transplanting the embryo into a pseudopregnant mammal, the blastocyst developing to term;
(c) identifying a mammal which carries a disruption in one allele, the mammal exhibiting a reduced severity of sepsis as compared to a wild type mammal.

34. A method for producing a homozygous non-human mammal having somatic and germ cells which contain a gene encoding a disrupted mammalian cIAP2 protein, according to claim 22, the method comprising:
(a) introducing the gene into an embryo blastocyst;
(b) transplanting the embryo into a pseudopregnant non-human mammal the blastocyst developing to term;
(c) identifying a first non-human mammal which carries the disrupted gene;
(d) inter-breeding the first mammal with a second non-human mammal carrying the disrupted gene; and
(e) identifying a non-human mammal which carries the disrupted gene in both alleles, the non-human mammal exhibiting a pathophysiological phenotype which is characterized by a substantial absence of expression of non human mammal cIAP2 protein, and by a reduced severity of sepsis.

35. A method of testing the transgenic non-human mammal, according to claim 24, for the severity of septic shock or endotoxic shock, the method comprising challenging the mammal with the shock and evaluating the effect of the severity of septic shock or endotoxic shock.

36. A vector comprising in the 5′ to 3′ direction a 5′ arm homologous to the cIAP2 gene; a marker sequence; and a 3′ arm homologous to the cIAP2 gene.

37. The vector, according to claim 36, in which the marker sequence comprises a splice acceptor site.

38. The vector, according to claim 36, in which the marker sequence comprises a bicistronic gene encoding beta-galactosidase.

39. The vector, according to claim 36, in which the marker sequence comprises an IRES-driven beta-galactosidase-neo fusion protein expression gene.

40. The vector, according to claim 39, in which the IRES-driven expression gene replaces exons 2 to 5 of cIAP2.

41. A cell comprising the vector, according to claim 36.

42. The cell, according to claim 41, which is a mouse embryonic stem cell.

43. A method for inducing apoptosis in a mammalian cell, the method comprising: administering directly to the cell the disrupted ciap2 gene, according to claim 22, so as to disrupt cIAP2 protein expression or function in the cell.

44. A method of screening compounds for treating sepsis or septic shock, the method comprising:
a) applying a sepsis or septic shock challenge to the transgenic non-human mammal, according to claim 24, the mammal exhibiting resistance to septic shock;
c) administering a test compound to the mammal;
d) determining the effect of the test compound on the severity of sepsis or septic shock in the mammal; and
e) correlating the effect of the test compound on septic shock or sepsis of the mammal with an effect of the test compound on the macrophages in a non treated mammal having a disrupted ciap2 gene, or in a wild type mammal.

45. A method of reducing mortality of sepsis, septic shock, endotoxic shock in a wild type mammal, the method comprising: comprising administering to the mammal in need thereof an antagonist of cIAP2 protein function and/or expression, thereby reducing the mortality of sepsis, septic shock, endotoxic shock

46. A method for reducing mortality of trauma in a wild type mammal, the method comprising: comprising administering to the mammal in need thereof an antagonist of cIAP2 protein function and/or expression, thereby reducing the mortality of trauma.

47. A plurality of cells derived from a transgenic non human animal, the cells comprising the disrupted ciap2 gene, according to claim 22.

48. A kit for determining the sensitivity of macrophages to apoptosis stimuli, the kit comprising a vial for receiving a sample of macrophages from the transgenic mammal, according to claim 24, after a septic shock or sepsis challenge; a stain for staining the macrophages for Annexin V; and instructions for comparing the stained macrophages with a control mammal.

49. A method for treating sepsis in a subject, the method comprising: providing in the subject in need thereof one or more cells, the cells being capable of producing progeny cells having disrupted cIAP2 protein expression and/or function, wherein the cells express the disrupted ciap2 gene, according to claim 22, under the control of a constitutive, inducible, or cell specific promoter so as tot cause apoptosis of the cells relative to untreated control cells not expressing cIAP2, thereby treating the sepsis.
